(19) United States
(12) Patent Application Publication
(10) Pub. No.: US 2020/0376007 A1
(43) Pub. Date: Dec. 3, 2020

(54) THERAPEUTIC COMPOUNDS AND METHODS OF USE THEREOF

(71) Applicant: REGENTS OF THE UNIVERSITY OF MINNESOTA, Minneapolis, MN (US)

(72) Inventors: Peter Ian Dosa, Minneapolis, MN (US); Alexanader Khoruts, Minneapolis, MN (US); Michael J. Sadowsky, Minneapolis, MN (US)

(73) Assignee: REGENTS OF THE UNIVERISTY OF MINNESOTA, Minneapolis, MN

(21) Appl. No.: 16/885,905

(22) Filed: May 28, 2020

Related U.S. Application Data
(60) Provisional application No. 62/854,253, filed on May 29, 2019.

(51) Int. Cl. A61K 31/58; A61K 31/575; A61P 3/10; A61P 3/04; A61P 1/00

(52) U.S. Cl. CPC A61K 31/58; A61K 31/575; A61P 1/00; A61P 3/04; A61P 3/10

(57) ABSTRACT

The invention provides a compound of formula (I):

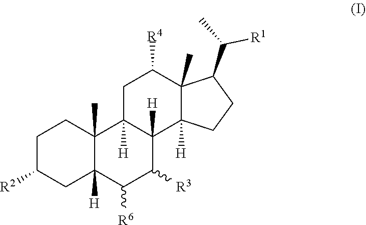

or a salt thereof, wherein R¹, R², R³, R⁴ and R⁶ have any of the values described in the specification, as well as compositions comprising a compound of formula (I). The compounds are agonists of the TGR5 receptor.

### PRIORITY

This application claims priority to U.S. Provisional Patent Application No. 62/854,253, filed 29 May 2019. The entire content of this United States Provisional Patent Application is hereby incorporated by reference herein.

### STATEMENT REGARDING FEDERALLY SPONSORED RESEARCH

This invention was made with government support under W81XWH-17-1-0635 and W81XWH-17-1-0636 awarded by Department of Defense. The government has certain rights in the invention.

### BACKGROUND

Bile acids are known for their role in aiding in the digestion and absorption of fats and fat-soluble vitamins and nutrients. The acids and their derivatives have been investigated as potential therapeutics for a surprisingly diverse set of clinical indications. Ursodeoxycholic acid (UDCA) and obeticholic acid, the 6α-ethyl derivative of chenodeoxycholic acid (CDCA), are both US-FDA approved drugs for the treatment of primary biliary cholangitis (Hirschfield, G. M. et al. Gastroenterology 2015, 148, 751-761). Obeticholic acid, an agonist of the farnesoid X nuclear receptor (FXR), also improved liver histology in clinical trials for non-alcoholic steatohepatitis (NASH) (Neuschwander-Tetri, B. A. et al. Gastroenterology 2015, 148, 704-706) and reduced portal hypertension in animal models (Len, V. et al. Hepatology 2014, 59, 2286-2298). UDCA derivatives, including the taurine conjugate TUDCA, have been extensively studied as anti-apoptotic agents, demonstrating beneficial effects in animal models of acute kidney injury, myocardial infarction, and stroke (Amaral, J. D. et al. J. Lipid Res. 2009, 50, 1721-1734). Other bile acid derivatives have been found to serve as glucocorticoid modulators (Sharma, R. et al. J. Med. Chem. 2011, 54, 122-130) or to inhibit the growth of human cancer cell lines (Ren, J. et al. Steroids 2013, 78, 53-58). Bile acids have also been used as carriers to improve the oral absorption of drugs, as bile acid-drug conjugates can potentially be actively absorbed from the gut by the same transporters that absorb endogenous bile acids, including the apical sodium-dependent bile acid transporter (ASBT) (Kramer, W. et al. Biol. Chem. 2011, 392, 77-94).

Importantly, bile acids are also endogenous agonists of the TGR5 receptor, alternatively referred to as GPBAR1 or M-BAR. The TGR5 receptor is widely expressed in human tissues, including the pancreas, liver, spleen, brain, skin, and the gastrointestinal tract (Duboc, H. et al. Dig. Liver Dis. 2014, 46, 302-312 van Nierop, F. S. et al. Lancet Diabetes Endocrinol. 2017, 5, 224-233 and Xu, Y. et al. J. Med. Chem. 2016, 59, 6553-6579). Both steroidal and non-steroidal TGR5 agonists have been studied extensively as possible therapeutics for type 2 diabetes and demonstrated significant improvements in GLP-1 secretion and glucose reduction in animal models of the disease (Xu, Y. et al. J. Med Chem. 2016, 59, 6553-6579). TGR5 activation is also believed to have anti-inflammatory effects (van Nierop, F. S. et al. Lancet Diabetes Endocrinol. 2017, 5, 224-233 and Baars. A. et al. Microorganisms 2015, 3, 641). However, TGR5 agonists are not without their potential drawbacks, leading to reports in animal models of problems with increased gallbladder filling (Li. T. et al. Mol. Endocrinol. 2011, 25, 1066-1071 and Briere, D. A. et al. PLoS ONE 2015, 10, e0136873), pruritus (Alemi, F. et al. J Clin. Invest. 2013, 123, 1513-1530), and significant reductions in vascular tone and blood pressure (Fryer, R. M. et al. J. Pharmacol. Exp. Ther. 2014, 348, 421-431).

Recent studies have investigated the interaction of bile acids and the TGR5 receptor as part of the ongoing research into finding improved methods for controlling Clostridium difficile infection (CDI) (Weingarden, A. R. et al. PLoS ONE 2016, 11, e0147210; Weingarden, A. R. et al. J. Clin. Gastroenterol. 2016, 50, 624-30 and Stoltz, K. L. et al. J. Med. Chem. 2017, 60, 3451-3471). C. difficile (recently reclassified as Clostridioides difficile) is an anaerobic, Gram-positive spore-forming bacterium. Spores of this bacterium use the presence of specific bile acids as an indicator of a favorable environment for germination. Current research has shown a number of synthetic UDCA and CDCA analogs capable of inhibiting the germination of C. difficile spores more potently than naturally occurring derivatives (Stoltz, K. L. et al. J. Med. Chem. 2017, 60, 3451-3471). In addition, a present chemical library contained a number of analogs in which the carboxylic acid of the parent bile acid was modified by conjugation. The availability of these analogs, along with several new derivatives that have been synthesized, provided an excellent opportunity to expand current knowledge of the structure-activity relationship (SAR) of bile acid derivatives at the TGR5 receptor. Interests in methods to increase the TGR5 potency of current derivatives, as TGR5 agonists could potentially reduce the symptoms of inflammatory bowel disease (Yoneno, K. et al. Immunology 2013, 139, 19-29), a condition often found alongside CDI that complicates the treatment of CDI by existing therapies including fecal microbiota transplantation (Khoruts, A. et al. Clin. Gastroenterol. Hepatol. 2016, 14, 1433-8).

US2014/0206657A1 relates to methods of synthesizing certain specific bile acid analogues and derivatives, their effects on TGR5 activity, and their possible use in treating diabetes and liver diseases.

WO2017/142895A1 relates to certain specific bile acid analogues and derivatives that are reported to be useful for preventing, treating, and/or reducing the risk of developing a Clostridium-associated disease.

Currently there is a need for additional agents that are agonists of the TGR5 receptor. In particular, there is a need for TGR5 agonists with improved properties, such as, for example, improved activity, potency, solubility, selectivity, or toxicity. Such agents would be useful for treating TGR5 mediated conditions, such as diabetes, obesity and/or inflammatory bowel disease.

### SUMMARY

In one aspect the present invention provides compounds that are potent agonists of the TGR5 receptor and useful for treating TGR5 mediated conditions (e.g. diabetes, obesity and/or inflammatory bowel disease).

Accordingly, the invention provides a method for treating a TGR5 mediated condition in an animal comprising administering to the animal a compound of formula (I):

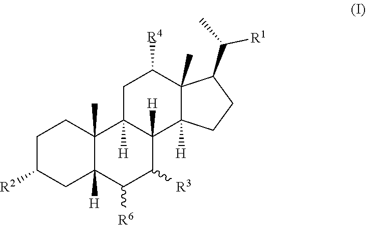

or a pharmaceutically acceptable salt thereof, wherein:

R¹ is (C₁-C₆)alkyl that is substituted with one or more R⁵, wherein R⁵ is independently selected from the group consisting of halo, cyano, heterocycle, heteroaryl, —OH, —C(═O)NH(C₁-C₆ alkyl)(S(O)₂R^{c}), —C(═O)NH(C₁-C₆ alkyl)(C(═O)OR^{c}), —C(═O)NR^{a}R^{b}, —OS(O)₃R^{c}, —C(═O)NH(S(O)₂R^{c}), —C(═O)OR^{c}, and —C(═O)(C₁-C₆ alkyl), wherein any heterocycle and heteroaryl is optionally substituted with one or more groups independently selected from the group consisting of halo, carboxy, —OH, cyano, (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy, wherein any (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy is optionally substituted with one or more groups independently selected from the group consisting of —OH and (C₁-C₆)alkoxy;

R² is selected from the group consisting of —OH, —OC(═O)R^{c}, —OS(O)₃H and (C₁-C₆)alkoxy;

R³ is selected from the group consisting of —OC(═O)R^{d}, (C₁-C₆)alkyl, —OS(O)₃R^{e} and (C₁-C₆)alkoxy;

R⁴ is selected from the group consisting of H and —OH, where;

R^{a} and R^{b} are independently selected from H and (C₁-C₆)alkyl that is optionally substituted with one or more groups independently selected from the group consisting of hydroxy and (C₁-C₆)alkoxy that is optionally substituted with one or more groups independently selected from the group consisting of hydroxy and (C₁-C₆)alkoxy; or R^{a} and R^{b} together with the nitrogen to which they are attached form a 4-6 membered ring heterocycle;

each R^{c} is independently selected from H and (C₁-C₆)alkyl;

R^{d} is H or (C₂-C₆)alkyl; or when R² is —OH, R^{d} is H or (C₁-C₆)alkyl;

R^{e} is (C₁-C₆)alkyl; and

R⁶ is selected from the group consisting of H and (C₁-C₆)alkyl, or R⁶ and R³ together with the atom to which they are attached form a carbocycle or heterocycle, wherein the carbocycle or heterocycle is optionally substituted with halo, cyano, heteroaryl, —OH and (C₁-C₆)alkyl.

The invention also provides a compound of formula (I) as or a pharmaceutically acceptable salt thereof, for the prophylactic or therapeutic treatment of a TGR5 mediated condition.

The invention also provides the use of a compound of formula (I) or a pharmaceutically acceptable salt thereof, to prepare a medicament for treating a TGR5 mediated condition.

The invention also provides a compound of formula (I):

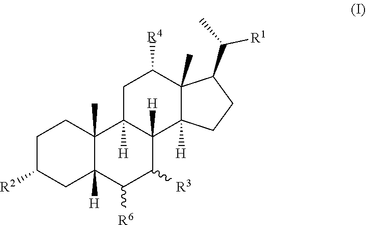

or a salt thereof, wherein:

R¹ is (C₁-C₆)alkyl that is substituted with one or more R⁵ wherein R⁵ is independently selected from the group consisting of halo, cyano, heterocycle, heteroaryl, —OH, —C(═O)NH(C₁-C₆ alkyl)(S(O)₂R^{c}), —C(═O)NH(C₁-C₆ alkyl)(C(═O)OR^{c}), —C(═O)NR^{a}R^{b}, —OS(O)₃R^{c}, —C(═O)NH(S(O)₂R^{c}), —C(═O)OR^{c}, and —C(═O)(C₁-C₆ alkyl), wherein any heterocycle and heteroaryl is optionally substituted with one or more groups independently selected from the group consisting of halo, carboxy, —OH, cyano, (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy, wherein any (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy is optionally substituted with one or more groups independently selected from the group consisting of —OH and (C₁-C₆)alkoxy;

R² is selected from the group consisting of —OH, —OC(═O)R^{c}, —OS(O)₃H and (C₁-C₆)alkoxy;

R³ is selected from the group consisting of —OC(═O)R^{d}, (C₁-C₆)alkyl, —OS(O)₃R^{c} and (C₁-C₆)alkoxy;

R⁴ is selected from the group consisting of H and —OH, where;

R^{a} and R^{b} are independently selected from H and (C₁-C₆)alkyl that is optionally substituted with one or more groups independently selected from the group consisting of hydroxy and (C₁-C₆)alkoxy that is optionally substituted with one or more groups independently selected from the group consisting of hydroxy and (C₁-C₆)alkoxy; or R^{a} and R^{b} together with the nitrogen to which they are attached form a 4-6 membered ring heterocycle;

each R^{c} is independently selected from H and (C₁-C₆)alkyl;

R^{d} is H or (C₂-C₆)alkyl; or when R² is —OH, R^{d} is H or (C₁-C₆)alkyl;

R^{d} is (C₁-C₆)alkyl;

R⁶ is selected from the group consisting of H and (C₁-C₆)alkyl, or R⁶ and R³ together with the atom to which they are attached form a carbocycle or heterocycle, wherein the carbocycle or heterocycle is optionally substituted with halo, cyano, heteroaryl, —OH and (C₁-C₆)alkyl;

provided that the compound or salt is not selected from the group consisting of:

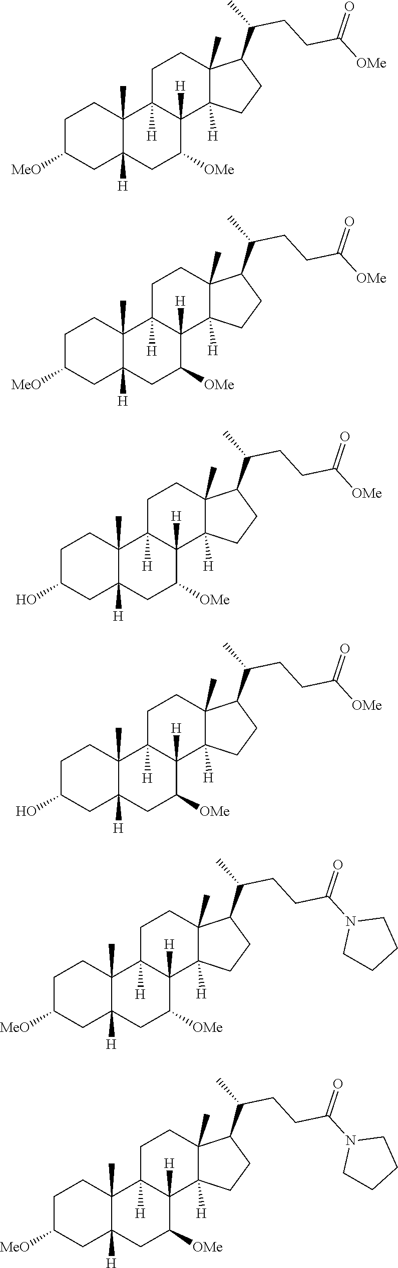

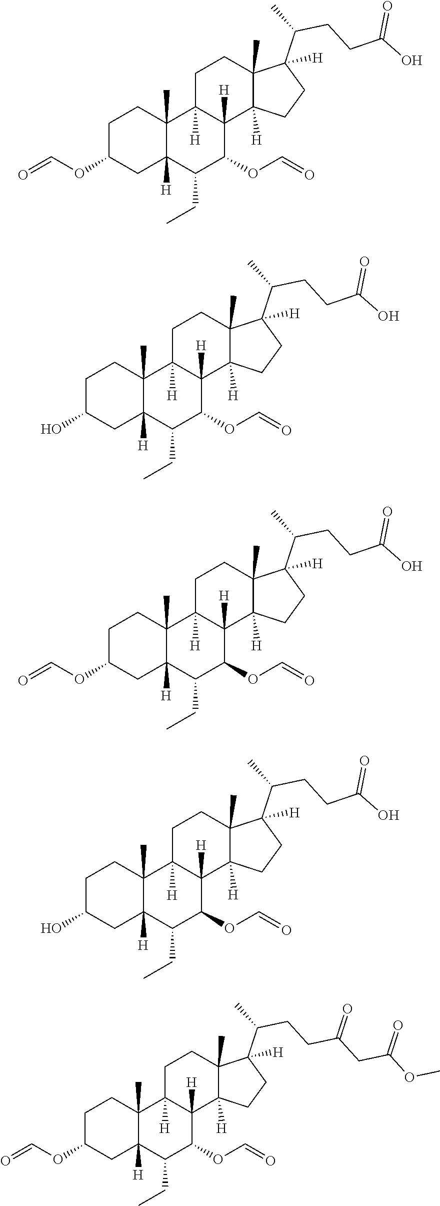

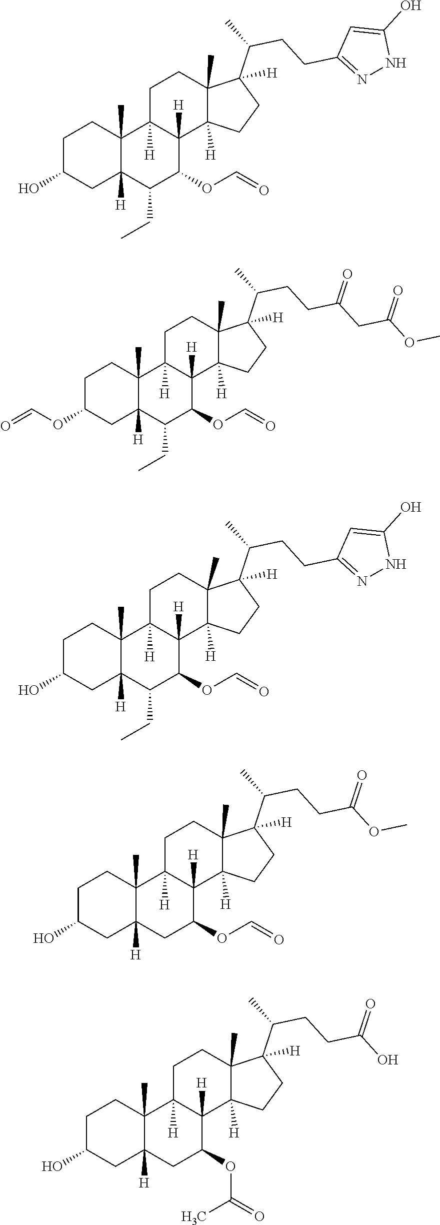

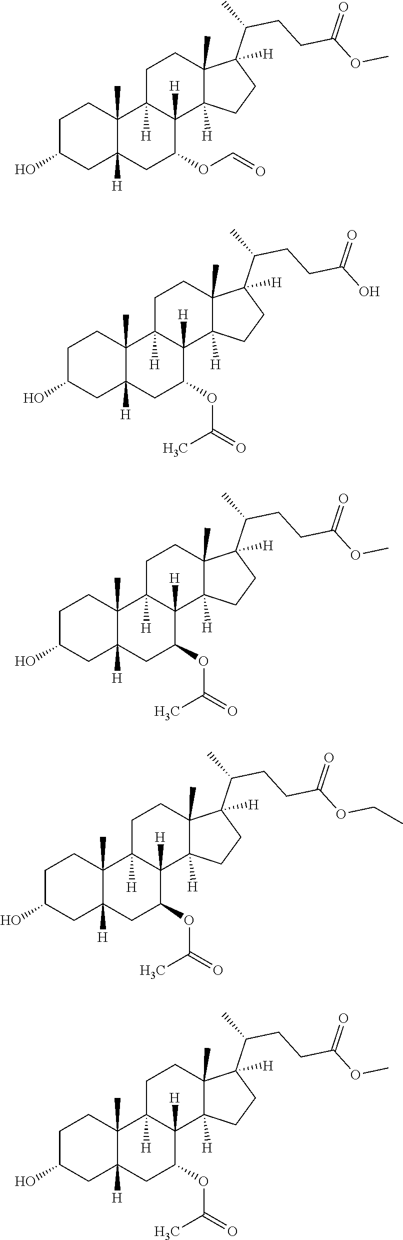

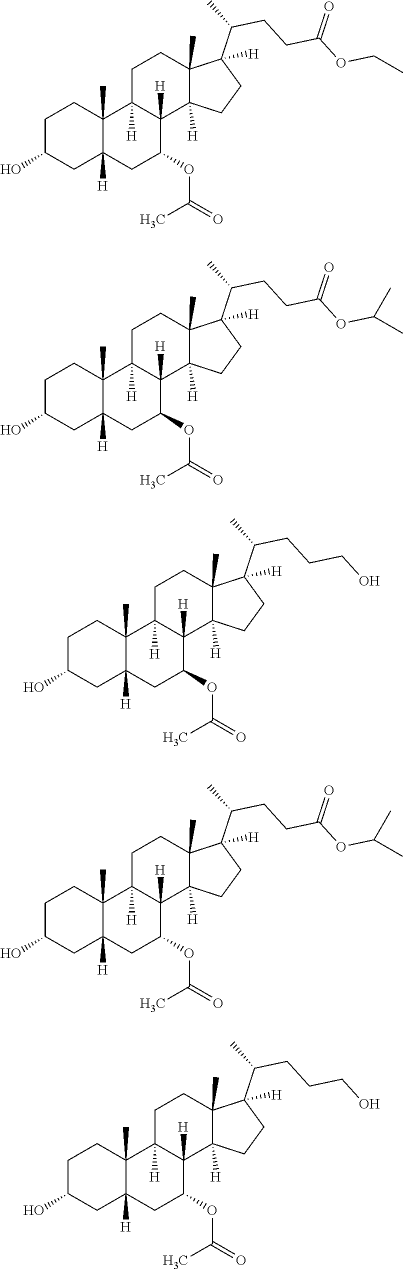

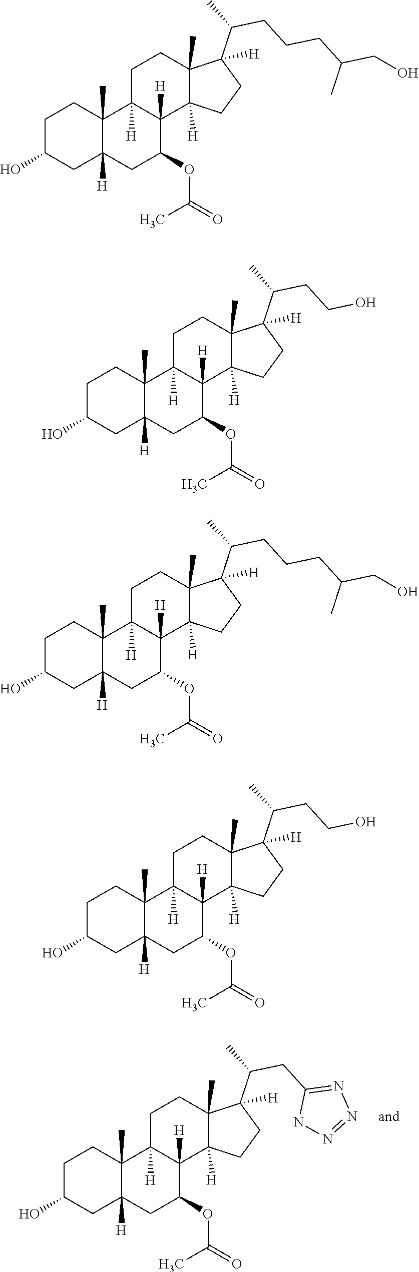

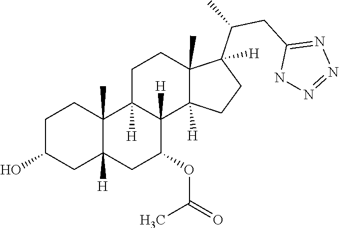

and salts thereof.

The invention also provides a pharmaceutical composition comprising a compound of formula (1) or a pharmaceutically acceptable salt thereof, and a pharmaceutically acceptable excipient.

The invention also provides a compound of formula (I) or a pharmaceutically acceptable salt thereof for use in medical therapy.

The invention also provides a method for promoting a TGR5 agonist effect comprising contacting TGR5 with an effective TGR5 agonising amount of a compound of formula (1) or a pharmaceutically acceptable salt thereof.

The invention also provides an anti-diabetic, anti-obesity, or anti-inflammatory bowel disease composition comprising a compound of formula (1) as or a pharmaceutically acceptable salt thereof.

The invention also provides processes and intermediates disclosed herein that are useful for preparing a compound of formula (I) or a salt thereof.

Applicant has discovered that C-7 alkoxy substitution in compounds of Formula (1) provides compounds with improved TGR5 agonist potency compared to corresponding compounds with C-7 hydroxy substitution. In one aspect, the invention is directed toward C-7 alkoxy (e.g. methoxy) substituted compounds and to their use in treating TGR5 mediated conditions, such as diabetes, inflammatory bowel disease, obesity, nonalcoholic steatohepatitis, primary bilary cholangitis, primary sclerosing cholangitis, gastric cancer, and/or other inflammatory diseases.

### BRIEF DESCRIPTION OF DRAWINGS

FIG. 1 shows structure of endogenous bile acids ursodeoxycholic acid (UDCA), tauroursodeoxycholic acid (TUDCA), chenodeoxycholic acid (CDCA), cholic acid, taurolithocholic acid (TLCA), and synthetic derivative obeticholic acid.

FIG. 2 shows Table 1: TGR5 Agonist Activity of Reference Compounds and Pyrrolidine Amides. ^{a}Data represent mean of at least two independent experiments. The ±standard error on the mean (SEM) is given when N≥3.

FIG. 3 shows Table 2: TGR5 Agonist Activity of CDCA Analogs. ^{a}Data represent mean of two independent experiments.

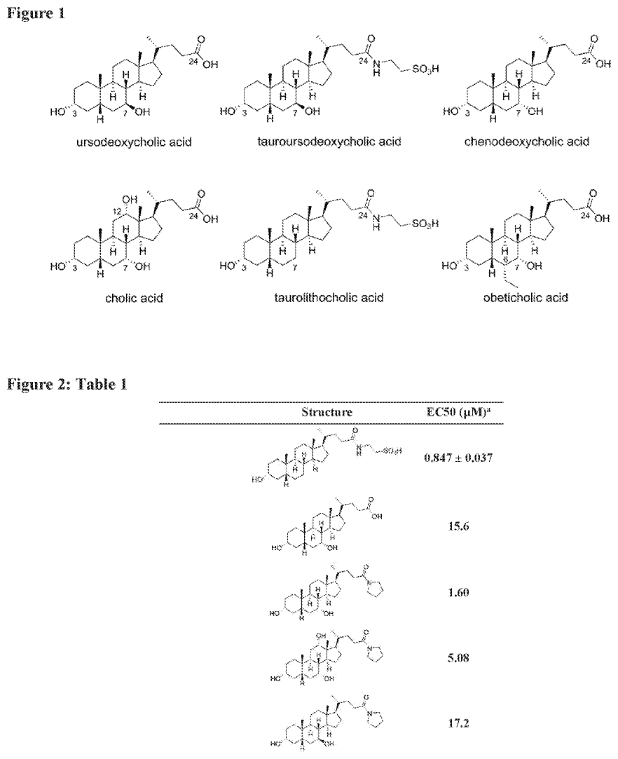

### DETAILED DESCRIPTION OF THE INVENTION

The following definitions are used, unless otherwise described: halo or halogen is fluoro, chloro, bromo, or iodo. Alkyl, alkoxy, alkenyl, alkynyl, etc. denote both straight and branched groups; but reference to an individual radical such as propyl embraces only the straight chain radical, a branched chain isomer such as isopropyl being specifically referred to.

The term “alkyl”, by itself or as part of another substituent, means, unless otherwise stated, a straight or branched chain hydrocarbon radical, having the number of carbon atoms designated (i.e., C₁₋₈ means one to eight carbons). Examples include (C₁-C₈)alkyl, (C₂-C₈)alkyl, C₁-C₆)alkyl, (C₂-C₆)alkyl and (C₃-C₆)alkyl. Examples of alkyl groups include methyl, ethyl, n-propyl, iso-propyl, n-butyl, t-butyl, iso-butyl, sec-butyl, n-pentyl, n-hexyl, n-heptyl, n-octyl, and and higher homologs and isomers.

The term “alkenyl” refers to an unsaturated alkyl radical having one or more double bonds. Examples of such unsaturated alkyl groups include vinyl, 2-propenyl, crotyl, 2-isopentenyl, 2-(butadienyl), 2,4-pentadienyl, 3-(1,4-pentadienyl) and the higher homologs and isomers.

The term “alkynyl” refers to an unsaturated alkyl radical having one or more triple bonds. Examples of such unsaturated alkyl groups ethynyl, 1- and 3-propynyl, 3-butynyl, and higher homologs and isomers.

The term “alkoxy” refers to an alkyl groups attached to the remainder of the molecule via an oxygen atom (“oxy”).

The term “alkylthio” refers to an alkyl groups attached to the remainder of the molecule via a thio group.

The term “cycloalkyl” refers to a saturated or partially unsaturated (non-aromatic) all carbon ring having 3 to 8 carbon atoms (i.e., (C₁-C₈)carbocycle). The term also includes multiple condensed, saturated all carbon ring systems (e.g., ring systems comprising 2, 3 or 4 carbocyclic rings). Accordingly, carbocycle includes multicyclic carbocyles such as a bicyclic carbocycles (e.g., bicyclic carbocycles having about 3 to 15 carbon atoms, about 6 to 15 carbon atoms, or 6 to 12 carbon atoms such as bicyclo[3.1.0]hexane and bicyclo[2.1.1]hexane), and polycyclic carbocycles (e.g tricyclic and tetracyclic carbocycles with up to about 20 carbon atoms). The rings of the multiple condensed ring system can be connected to each other via fused, spiro and bridged bonds when allowed by valency requirements. For example, multicyclic carbocyles can be connected to each other via a single carbon atom to form a spiro connection (e.g., spiropentane, spiro[4,5]decane, etc), via two adjacent carbon atoms to form a fused connection (e.g., carbocycles such as decahydronaphthalene, norsabinane, norcarane) or via two non-adjacent carbon atoms to form a bridged connection (e.g., norbornane, bicyclo[2.2.2]octane, etc). Non-limiting examples of cycloalkyls include cyclopropyl, cyclobutyl, cyclopentyl, cyclohexyl, bicyclo[2.2.1]heptane, pinane, and adamantane.

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed carbon ring systems (e.g., ring systems comprising 2, 3 or 4 rings) having about 9 to 20 carbon atoms in which at least one ring is aromatic and wherein the other rings may be aromatic or not aromatic (i.e., cycloalkyl. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. Non-limiting examples of aryl groups include, but are not limited to, phenyl, indenyl, indanyl, naphthyl, 1, 2, 3, 4-tetrahydronaphthyl, anthracenyl, and the like.

The term “heterocycle” refers to a single saturated or partially unsaturated ring that has at least one atom other than carbon in the ring, wherein the atom is selected from the group consisting of oxygen, nitrogen and sulfur; the term also includes multiple condensed ring systems that have at least one such saturated or partially unsaturated ring, which multiple condensed ring systems are further described below. Thus, the term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The sulfur and nitrogen atoms may also be present in their oxidized forms. Exemplary heterocycles include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more groups selected from cycloalkyl, aryl, and heterocycle to form the multiple condensed ring system. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heterocycle) can be at any position of the multiple condensed ring system including a heterocycle, aryl and carbocycle portion of the ring. In one embodiment the term heterocycle includes a 3-15 membered heterocycle. In one embodiment the term heterocycle includes a 3-10 membered heterocycle. In one embodiment the term heterocycle includes a 3-8 membered heterocycle. In one embodiment the term heterocycle includes a 3-7 membered heterocycle. In one embodiment the term heterocycle includes a 3-6 membered heterocycle. In one embodiment the term heterocycle includes a 4-6 membered heterocycle. In one embodiment the term heterocycle includes a 3-10 membered monocyclic or bicyclic heterocycle comprising 1 to 4 heteroatoms. In one embodiment the term heterocycle includes a 3-8 membered monocyclic or bicyclic heterocycle comprising 1 to 3 heteroatoms. In one embodiment the term heterocycle includes a 3-6 membered monocyclic heterocycle comprising 1 to 2 heteroatoms. In one embodiment the term heterocycle includes a 4-6 membered monocyclic heterocycle comprising 1 to 2 heteroatoms. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl dihydrooxazolyl, chromanyl, 1,2-dihydropyridinyl, 2,3-dihydrobenzofuranyl, 1,3-benzodioxolyl, 1,4-benzodioxanyl, spiro[cyclopropane-1,1-isoindolinyl]-3′-one, isoindolinyl-1-one, 2-oxa-6-azaspiro[3.3]heptanyl, imidazolidin-2-one imidazolidine, pyrazolidine, butyrolactam, valerolactam, imidazolidinone, hydantoin, dioxolane, phthalimide, and 1,4-dioxane.

The term “heteroaryl” as used herein refers to a single aromatic ring that has at least one atom other than carbon in the ring, wherein the atom is selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl oxazolyl or furyl. “Heteroaryl” also includes multiple condensed ring systems (e.g. ring systems comprising 2, 3 or 4 rings) wherein a heteroaryl group, as defined above, is condensed with one or more rings selected from cycloalkyl, aryl, heterocycle, and heteroaryl. It is to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, triazolyl, tetrazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, and quinazolyl.

The term “alkoxycarbonyl” as used herein refers to a group (alkyl)-O—C(═O)—, wherein the term alkyl has the meaning defined herein.

The term “alkanoyloxy” as used herein refers to a group (alkyl)-C(═O)—O—, wherein the term alkyl has the meaning defined herein.

As used herein, the term “heteroatom” is meant to include oxygen (O), nitrogen (N), sulfur (S) and silicon (Si).

As used herein, the term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl 2-(trimethylsilyl)ethoxymethyl, 2-4p-toluenesulfonyl)ethyl, 2-(p-nitrophenylsulfenyl)ethyl, 2-(diphenylphosphino)-ethyl, nitroethyl and the like. For a general description of protecting groups and their use, see P. G. M. Wuts and T. W. Greene, Greene's Protective Groups in Organic Synthesis 4^{th} edition, Wiley-Interscience, New York, 2006.

As used herein a wavy line “ [symbol] ” that intersects a bond in a chemical structure indicates the point of attachment of the bond that the wavy bond intersects in the chemical structure to the remainder of a molecule.

The terms “treat” “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the object is to prevent or slow down (lessen) an undesired physiological change or disorder, such as, for example, the development or spread of cancer. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treat”, “treatment”, or “treating,” can also mean prolonging survival as compared to expected survival if not receiving treatment. Those in need of treatment include those already with the disease or disorder as well as those prone to have the disease or disorder or those in which the disease or disorder is to be prevented. In one embodiment “treat”, “treatment”, or “treating” does not include preventing or prevention,

The phrase “therapeutically effective amount” or “effective amount” includes but is not limited to an amount of a compound of the that (i) treats or prevents the particular disease, condition, or disorder, (ii) attenuates, ameliorates, or eliminates one or more symptoms of the particular disease, condition, or disorder, (iii) prevents or delays the onset of one or more symptoms of the particular disease, condition, or disorder described herein, or (iv) produces the described biological effect (e.g. produces a TGR5 agonist effect).

The term “mammal” as used herein refers to humans, higher non-human primates, rodents, domestic, cows, horses, pigs, sheep, dogs and cats. In one embodiment, the mammal is a human. The term “patient” as used herein refers to any animal including mammals. In one embodiment, the patient is a mammalian patient. In one embodiment, the patient is a human patient.

The compounds disclosed herein can also exist as tautomeric isomers in certain cases. Although only one delocalized resonance structure may be depicted, all such forms are contemplated within the scope of the invention

It is understood by one skilled in the art that this invention also includes any compound claimed that may be enriched at any or all atoms above naturally occurring isotopic ratios with one or more isotopes such as, but not limited to, deuterium (2H or D). As a non-limiting example, a —CH₃ group may be substituted with —CD₃.

The pharmaceutical compositions of the invention can comprise one or more excipients. When used in combination with the pharmaceutical compositions of the invention the term “excipients” refers generally to an additional ingredient that is combined with the compound of formula (I) or the pharmaceutically acceptable salt thereof to provide a corresponding composition. For example, when used in combination with the pharmaceutical compositions of the invention the term “excipients” includes, but is not limited to: carriers, binders, disintegrating agents, lubricants, sweetening agents, flavoring agents, coatings, preservatives, and dyes.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

The term “residue” as it applies to the residue of a compound refers to a compound that has been modified in any manner which results in the creation of an open valence wherein the site of the open valence. The open valence can be created by the removal of 1 or more atoms from the compound (e.g., removal of a single atom such as hydrogen or removal of more than one atom such as a group of atoms including but not limited to an amine, hydroxyl, methyl, amide (e.g., —C(═O)NH₂) or acetyl group). The open valence can also be created by the chemical conversion of a first function group of the compound to a second functional group of the compound (e.g., reduction of a carbonyl group, replacement of a carbonyl group with an amine) followed by the removal of 1 or more atoms from the second functional group to create the open valence.

Specific values listed below for radicals, substituents, and ranges, are for illustration only; they do not exclude other defined values or other values within defined ranges for the radicals and substituents. It is to be understood that two or more values may be combined. It is also to be understood that the values listed herein below (or subsets thereof) can be excluded.

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, I-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

In one embodiment, the compound of formula (1) is a compound of formula (Ia):

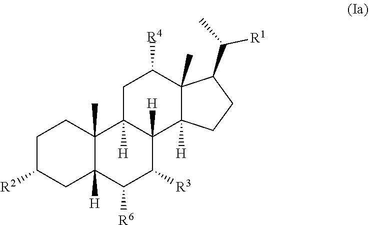

or a pharmaceutically acceptable salt thereof, wherein:

R¹ is (C₁-C₆)alkyl that is substituted with one or more R⁵, wherein R⁵ is independently selected from the group consisting of halo, cyano, heterocycle, heteroaryl, —OH, —C(═O)NH(C₁-C₆ alkyl)(S(O)₂R^{c}), —C(═O)NH(C₁-C₆ alkyl)(C(═O)OR^{c}), —C(═O)NR^{a}R^{b}, —OS(O))₃R^{c}, —C(═O)NH(S(O)₂R^{c}), —C(═O)OR^{c}, and —C(═O)(C₁-C₆ alkyl), wherein any heterocycle and heteroaryl is optionally substituted with one or more groups independently selected from the group consisting of halo, carboxy, —OH, cyano, (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy, wherein any (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy is optionally substituted with one or more groups independently selected from the group consisting of —OH and (C₁-C₆)alkoxy;

R² is selected from the group consisting of —OH, —OC(═O)R^{c}, —OS(O)₃H and (C₁-C₆)alkoxy;

R³ is selected from the group consisting of —OC(═O)R^{d}, (C₁-C₆)alkyl, —OS(O)₃R^{c} and (C₁-C₆)alkoxy;

R⁴ is selected from the group consisting of H and —OH, where;

R^{a} and R^{b} are independently selected from H and (C₁-C₆)alkyl that is optionally substituted with one or more groups independently selected from the group consisting of hydroxy and (C₁-C₆)alkoxy that is optionally substituted with one or more groups independently selected from the group consisting of hydroxy and (C₁-C₆)alkoxy; or R^{a} and R^{b} together with the nitrogen to which they are attached form a 4-6 membered ring heterocycle;

each R^{c} is independently selected from H and (C₁-C₆)alkyl;

R^{d} is H or (C₂-C₆)alkyl; or when R² is —OH, R^{d} is H or (C₁-C₆)alkyl;

R^{e} is (C₁-C₆)alkyl and

R⁶ is selected from the group consisting of H and (C₁-C₆)alkyl.

In one embodiment, the compound of formula (1) is a compound of formula (Ib):

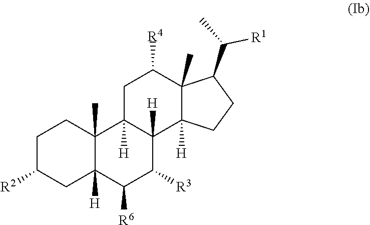

or a pharmaceutically acceptable salt thereof, wherein:

R¹ is (C₁-C₆)alkyl that is substituted with one or more R⁵, wherein R⁵ is independently selected from the group consisting of halo, cyano, heterocycle, heteroaryl, —OH, —C(═O)NH(C₁-C₆ alkyl)(S(O)₂R^{c}), —C(═O)NH(C₁-C₆ alkyl)(C(═O)OR), —C(═O)NR^{a}R^{b}, —OS(O)₃R^{c}, —C(═O)NH(S(O)₂R^{c}), —C(═O)OR^{c}, and —C(═)(C₁-C₆ alkyl), wherein any heterocycle and heteroaryl is optionally substituted with one or more groups independently selected from the group consisting of halo, carboxy, —OH, cyano, (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy, wherein any (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxy carbonyl, (C₁-C₆)alkylthio and (C₂-C₆)alkanoyloxy is optionally substituted with one or more groups independently selected from the group consisting of —OH and (C₁-C₆)alkoxy;

R² is selected from the group consisting of —OH, —OC(═O)R^{c}, —OS(O)₃H and (C₁-C₆)alkoxy;

R³ is selected from the group consisting of —OC(═O)R^{d}, (C₁-C₆)alkyl, —OS(O)₃R^{e} and (C₁-C₆)alkoxy;

R⁴ is selected from the group consisting of H and —OH, where;

R^{a} and R^{b} are independently selected from H and (C₁-C₆)alkyl that is optionally substituted with one or more groups independently selected from the group consisting of hydroxy and (C₁-C₆)alkoxy that is optionally substituted with one or more groups independently selected from the group consisting of hydroxy and (C₁-C₆)alkoxy; or R^{a} and R^{b} together with the nitrogen to which they are attached form a 4-6 membered ring heterocycle;

each R^{c} is independently selected from H and (C₁-C₆)alkyl;

R^{d} is H or (C₂-C₆)alkyl; or when R² is —OH, R^{d} is H or (C₁-C₆)alkyl;

R^{e} is (C₁-C₆)alkyl; and

R⁶ is selected from the group consisting of H and (C₁-C₆)alkyl.

In one embodiment, the compound of formula (I) is a compound of formula (Ic):

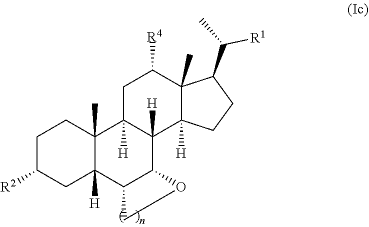

or a pharmaceutically acceptable salt thereof, wherein:

R¹ is (C₁-C₆)alkyl that is substituted with one or more R⁵, wherein R⁵ is independently selected from the group consisting of halo, cyano, heterocycle, heteroaryl, —OH, —C(═O)NH(C₁-C₆ alkyl)(S(O)₂R^{c}), —C(═O)NH(C₁-C₆ alkyl)(C(═O)OR), —C(═O)NR^{a}R^{b}, —OS(O)₃R^{c}, —C(═O)NH(S(O)₂R^{c}), —C(═O)OR^{c}, and —C(═)(C₁-C₆ alkyl), wherein any heterocycle and heteroaryl is optionally substituted with one or more groups independently selected from the group consisting of halo, carboxy, —OH, cyano, (C₁-C₆)alkyl, (C₁-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy, wherein any (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy is optionally substituted with one or more groups independently selected from the group consisting of —OH and (C₁-C₆)alkoxy;

R² is selected from the group consisting of —OH, —OC(═O)R^{c}, —OS(O)₃H and (C₁-C₆)alkoxy;

R⁴ is selected from the group consisting of H and —OH, where;

R^{a} and R^{b} are independently selected from H and (C₁-C₆)alkyl that is optionally substituted with one or more groups independently selected from the group consisting of hydroxy and (C₁-C₆)alkoxy that is optionally substituted with one or more groups independently selected from the group consisting of hydroxy and (C₁-C₆)alkoxy; or R^{a} and R^{b} together with the nitrogen to which they are attached form a 4-6 membered ring heterocycle;

each R^{c} is independently selected from H and (C₁-C₆)alkyl;

R^{e} is (C₁-C₆)alkyl; and

n is 1, 2, or 3.

In one embodiment, R¹ is (C₃-C₆)alkyl that is substituted with one or more R⁵, wherein R⁵ is independently selected from the group consisting of halo, cyano, heterocycle, heteroaryl, —OH, —C(═O)NH(C₁-C₆ alkyl)(S(O)₂R^{c}), —C(═O)NH(C₁-C₆ alkyl)(C(═O)OR^{c}), —C(═O)NR^{a}R^{b}, —OS(O)₃R^{c}, —C(═O)NH(S(O)₂R^{c}), —C(═O)OR^{c}, and —C(═O)(C₁-C₆ alkyl), wherein any heterocycle and heteroaryl is optionally substituted with one or more groups independently selected from the group consisting of halo, carboxy, —OH, cyano, (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy, wherein any (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy is optionally substituted with one or more groups independently selected from the group consisting of —OH and (C₁-C₆)alkoxy.

In one embodiment, R¹ is (C₁-C₆)alkyl that is substituted with one or more R⁵, wherein R⁵ is independently selected from the group consisting of halo, cyano, heterocycle, heteroaryl, —OH, —C(═)NH(C₁-C₆ alkyl)(S(O)₂R^{c}), —C(═O)NH(C₁-C₆ alkyl)(C(═O)OR^{c}), —C(═O)NR^{a}R^{b}, —OS(O)₃R^{c}, —C(═O)NH(S(O)₂R^{c}), —C(═O)OR^{c}, and —C(═O)(C₁-C₆ alkyl), wherein any heterocycle and heteroaryl is optionally substituted with one or more groups independently selected from the group consisting of halo, carboxy, —OH, cyano, (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy, wherein any (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy is optionally substituted with one or more groups independently selected from the group consisting of —OH and (C₁-C₆)alkoxy.

In one embodiment, R¹ is

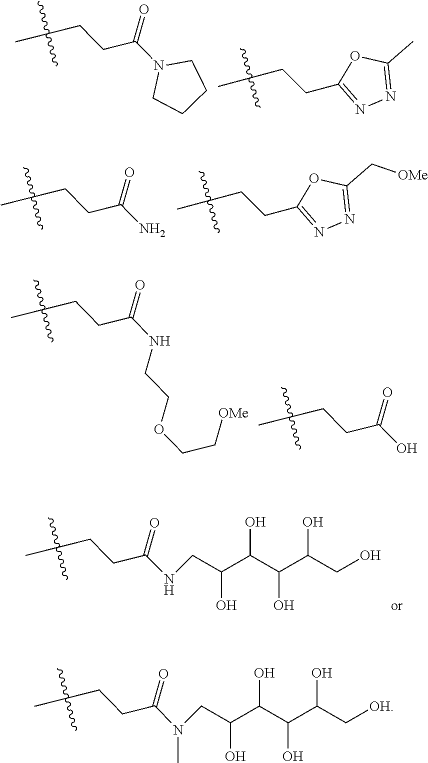

In one embodiment, R¹ is

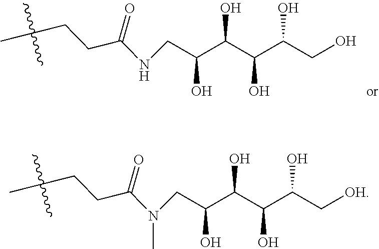 [SMILES: *CCC(=O)NC[C@H](O)[C@@H](O)[C@H](O)[C@H](O)CO ; *CCC(=O)N(C)C[C@H](O)[C@@H](O)[C@H](O)[C@H](O)CO]

In one embodiment, R¹ is

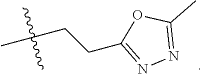 [SMILES: *CCc1nnc(C)o1]

In one embodiment, R⁵ is heteroaryl optionally substituted with (C₁-C₆)alkyl.

In one embodiment, R⁵ is heteroaryl substituted with methyl.

In one embodiment, R⁵ is 2-methyl-1,3,4-oxadiazolyl.

In one embodiment, R⁵ is —C(═O)NH₂.

In one embodiment, R⁵ is —C(═O)NR^{a}R^{b}, wherein R^{a} and R^{b} together form pyrrolidinyl.

In one embodiment, R⁵ is —C(O)NH(S(O)₂CH₃).

In one embodiment, R⁵ is —C(═O)NH(CH₂)₂S(O)₂H.

In one embodiment, R⁵ is —C(═O)NHCH₂CO₂H.

In one embodiment, R⁵ is —OS(O)₃H.

In one embodiment, R⁵ is —CO₂H.

In one embodiment, R² is —OC(═O)H.

In one embodiment, R¹ is —OMe.

In one embodiment, R² is —OH.

In one embodiment, R² is —OH, —OMe, or —OC(═O)H.

In one embodiment, R³ is —OMe.

In one embodiment, R³ is —OEt.

In one embodiment, R³ is —OMe or —OC(═O)H.

In one embodiment, R⁴ is H.

In one embodiment, R⁴ is —OH.

In one embodiment, R⁶ is H.

In one embodiment, R^{d} is (C₂-C₆)alkyl.

In one embodiment, the invention provides a compound of formula (I):

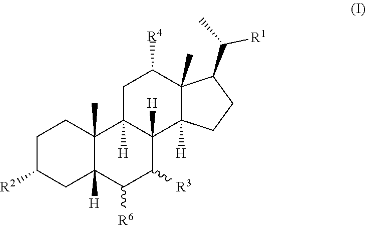

or a salt thereof, wherein:

R¹ is (C₁-C₆)alkyl that is substituted with one or more R⁵, wherein R⁵ is independently selected from the group consisting of halo, cyano, heterocycle, heteroaryl, —OH, —C(═O)NH(C₁-C₆ alkyl)(S(O)₂R^{c}), —C(═O)NH(C₁-C₆ alkyl)(C(═O)OR^{c}), —C(═O)NR^{a}R^{b}, —OS(O)₃R^{c}, —C(═O)NH(S(O)₂R^{c}), —C(═O)OR^{c}, and —C(═O)(C₁-C₆ alkyl), wherein any heterocycle and heteroaryl is optionally substituted with one or more groups independently selected from the group consisting of halo, carboxy, —OH, cyano, (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy, wherein any (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy is optionally substituted with one or more groups independently selected from the group consisting of —OH and (C₁-C₆)alkoxy;

R² is selected from the group consisting of —OH, —OC(═O)R^{c}, —OS(O)₃H and (C₁-C₆)alkoxy;

R³ is (C₁-C₆)alkyl;

R⁴ is selected from the group consisting of H and —OH, where;

R^{a} and R^{b} are independently selected from H and (C₁-C₆)alkyl that is optionally substituted with one or more groups independently selected from the group consisting of hydroxy and (C₁-C₆)alkoxy that is optionally substituted with one or more groups independently selected from the group consisting of hydroxy and (C₁-C₆)alkoxy; or R^{a} and R^{b} together with the nitrogen to which they are attached form a 4-6 membered ring heterocycle;

each R^{c}, is independently selected from H and (C₁-C₆)alkyl;

R^{e} is (C₁-C₆)alkyl; and

R⁶ is selected from the group consisting of H and (C₁-C₆)alkyl, or R⁶ and R³ together with the atom to which they are attached form a carbocycle or heterocycle, wherein the carbocycle or heterocycle is optionally substituted with halo, cyano, heteroaryl, —OH and (C₁-C₆)alkyl.

In one embodiment, the invention provides a compound of formula (I):

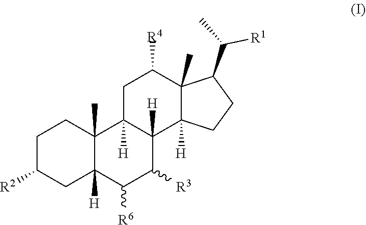

or a salt thereof, wherein:

R¹ is (C₁-C₆)alkyl that is substituted with one or more R⁵, wherein R⁵ is independently selected from the group consisting of halo, cyano, heterocycle, heteroaryl, —OH, —C(═O)NH(C₁-C₆ alkyl)(S(O)₂R^{c}), —C(═O)NH(C₁-C₆ alkyl)(C(═O)OR^{c}), —C(═)NR^{a}R^{b}, —OS(O)₃R^{c}, —C(═O)NH(S(O)₂R^{c}), —C(═O)OR^{c}, and —C(═O)(C₁-C₆ alkyl), wherein any heterocycle and heteroaryl is optionally substituted with one or more groups independently selected from the group consisting of halo, carboxy, —OH, cyano, (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy, wherein any (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy is optionally substituted with one or more groups independently selected from the group consisting of —OH and (C₁-C₆)alkoxy;

R² is selected from the group consisting of —OH, —OC(═O)R^{c}, —OS(O)₃H and (C₁-C₆)alkoxy;

R³ is (C₁-C₆)alkoxy;

R⁴ is selected from the group consisting of H and —OH, where;

R^{a} and R^{b} are independently selected from H and (C₁-C₆)alkyl that is optionally substituted with one or more groups independently selected from the group consisting of hydroxy and (C₁-C₆)alkoxy that is optionally substituted with one or more groups independently selected from the group consisting of hydroxy and (C₁-C₆)alkoxy; or R^{a} and R^{b} together with the nitrogen to which they are attached form a 4-6 membered ring heterocycle;

each R^{c} is independently selected from H and (C₁-C₆)alkyl;

R^{e} is (C₁-C₆)alkyl;

R⁶ is selected from the group consisting of H and (C₁-C₆)alkyl, or R⁶ and R³ together with the atom to which they are attached form a carbocycle or heterocycle, wherein the carbocycle or heterocycle is optionally substituted with halo, cyano, heteroaryl, —OH and (C₁-C₆)alkyl;
provided that the compound or salt is not selected from the group consisting of:

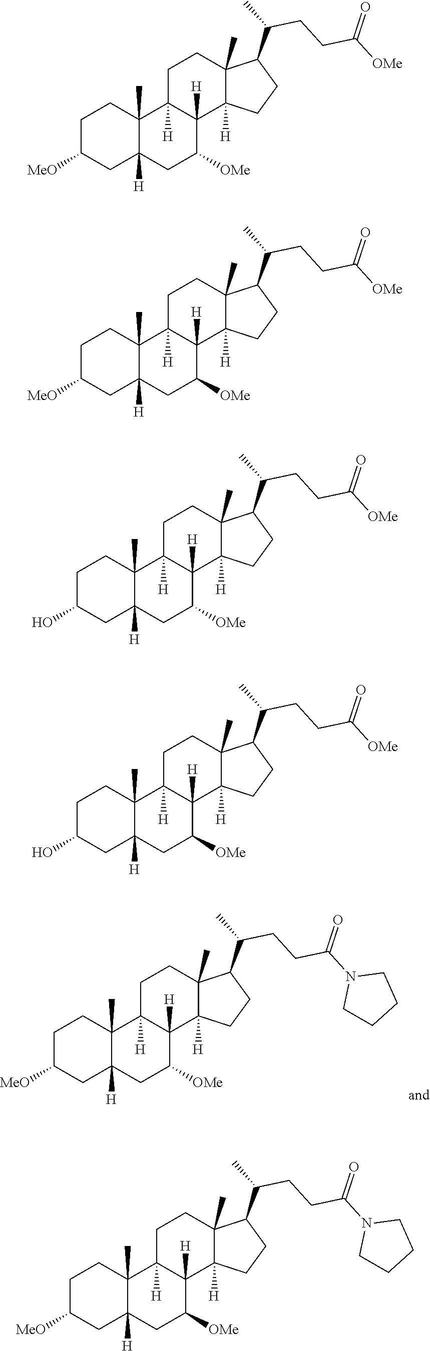

and salts thereof.

In one embodiment, the invention provides a compound of formula (1):

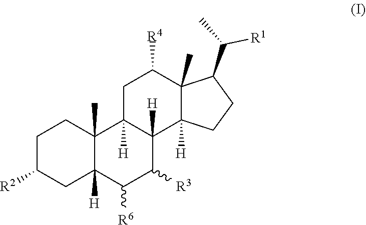

or a salt thereof, wherein:

R¹ is (C₁-C₆)alkyl that is substituted with one or more R⁵, wherein R⁵ is independently selected from the group consisting of halo, cyano, heterocycle, heteroaryl, —OH, —C(═O)NH(C₁-C₆ alkyl)(S(O)₂R^{c}), —C(═O)NH(C₁-C₆ alkyl)(C(═O)O^{c}), —C(═O)NR^{a}R^{b}, —OS(O)₃R^{c}, —C(═O)NH(S(O)₂R^{c}), —C(═O)OR^{c}, and —C(═O)(C₁-C₆ alkyl), wherein any heterocycle and heteroaryl is optionally substituted with one or more groups independently selected from the group consisting of halo, carboxy, —OH, cyano, (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy, wherein any (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy is optionally substituted with one or more groups independently selected from the group consisting of —OH and (C₁-C₆)alkoxy;

R² is selected from the group consisting of —OH, —OC(═O)R^{c}, —OS(O)₃H and (C₁-C₆)alkoxy;

R³ is —OC(═O)R^{d};

R⁴ is selected from the group consisting of H and —OH, where;

R^{a} and R^{b} are independently selected from H and (C₁-C₆)alkyl that is optionally substituted with one or more groups independently selected from the group consisting of hydroxy and (C₁-C₆)alkoxy that is optionally substituted with one or more groups independently selected from the group consisting of hydroxy and (C₁-C₆)alkoxy or R^{a} and R^{b} together with the nitrogen to which they are attached form a 4-6 membered ring heterocycle;

each R is independently selected from H and (C₁-C₆)alkyl;

R^{d} is H or (C₂-C₆)alkyl; or when R² is —OH, R^{d} is H or (C₁-C₆)alkyl;

R^{e} is (C₁-C₆)alkyl; and

R⁶ is selected from the group consisting of H and (C₁-C₆)alkyl, or R⁶ and R³ together with the atom to which they are attached form a carbocycle or heterocycle, wherein the carbocycle or heterocycle is optionally substituted with halo, cyano, heteroaryl, —OH and (C₁-C₆)alkyl;
provided that the compound or salt is not selected from the group consisting of:

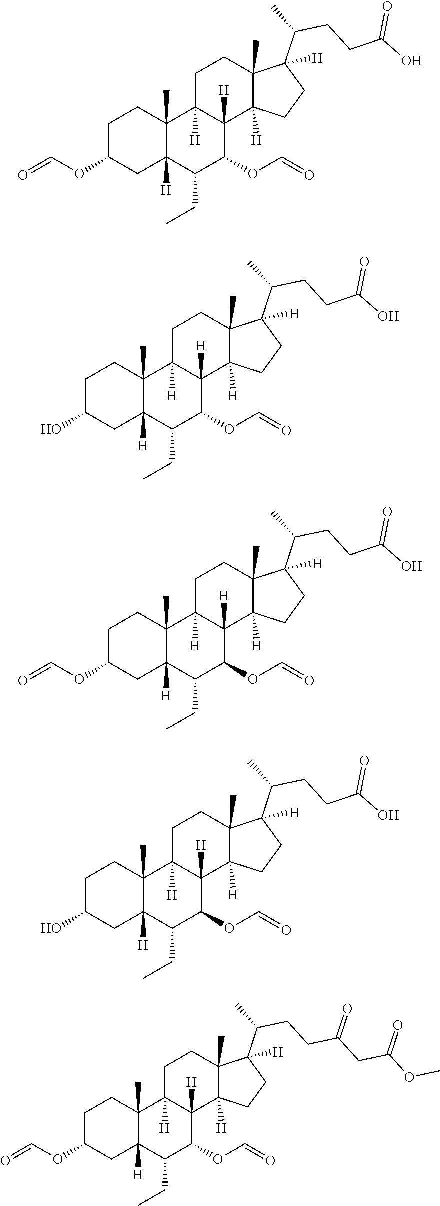

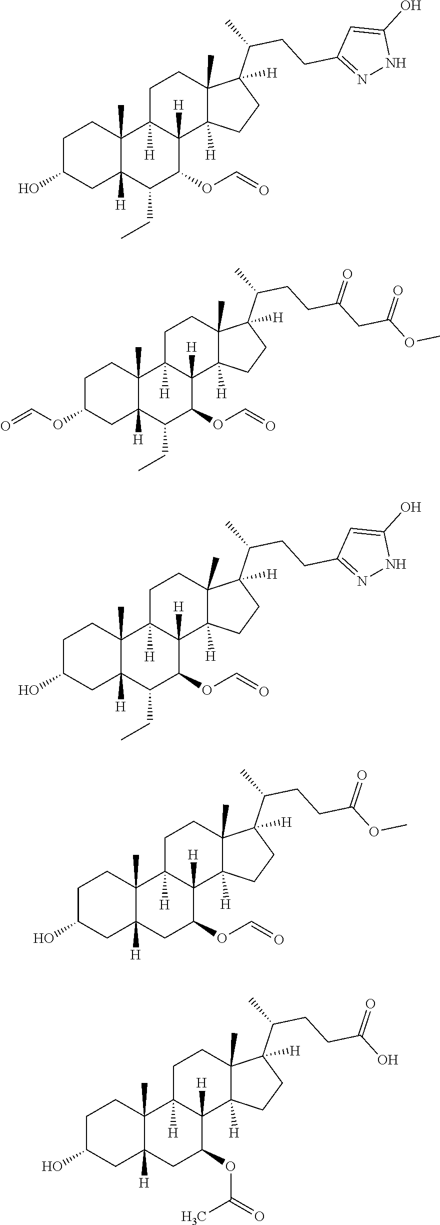

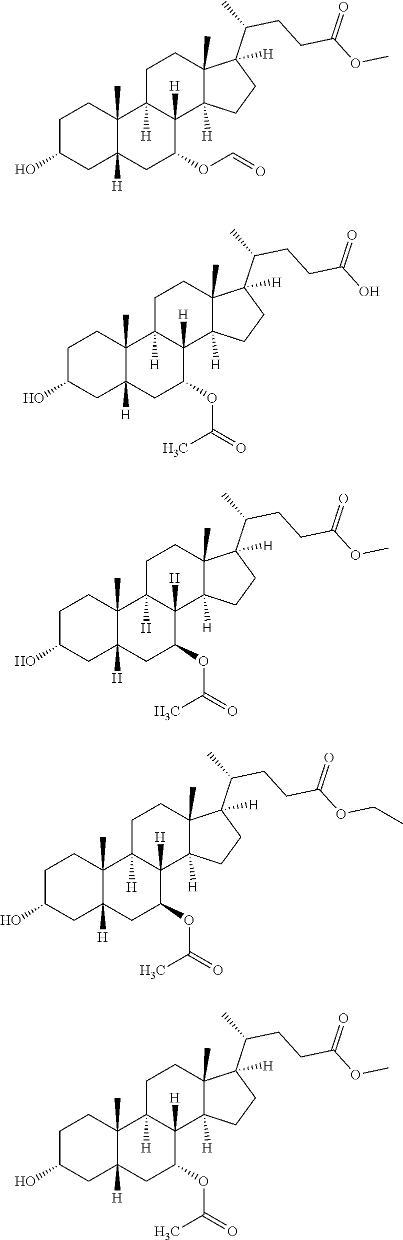

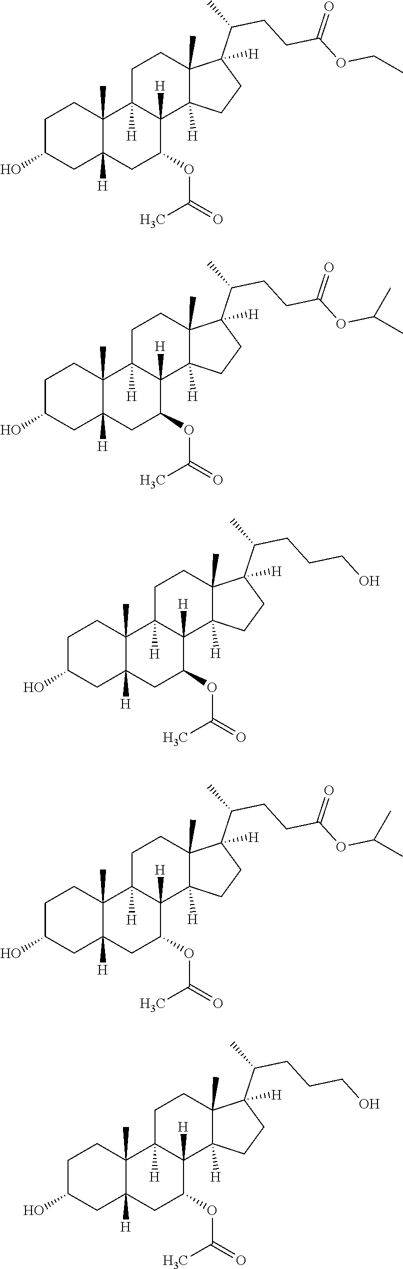

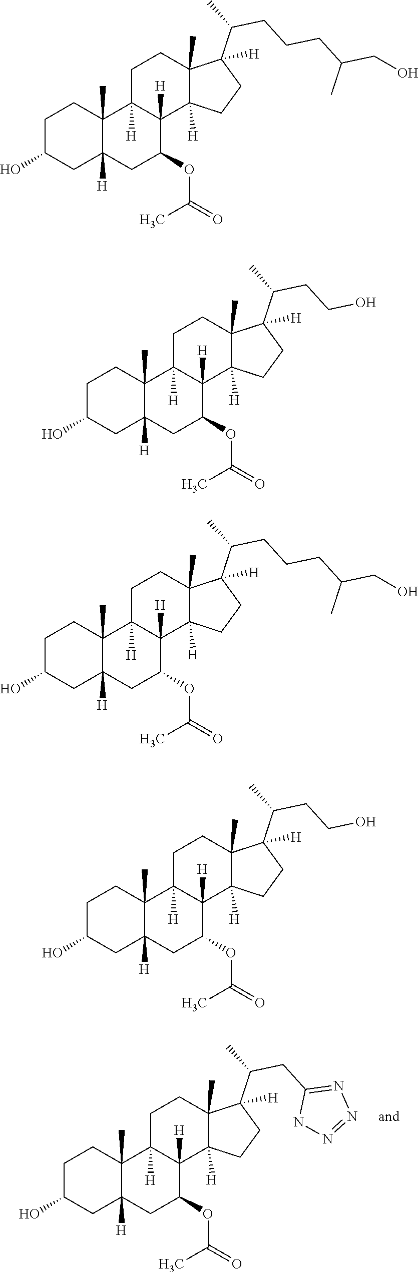

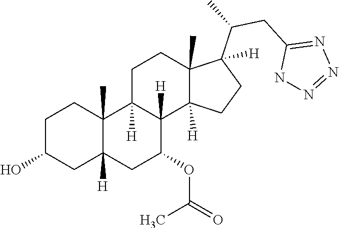

and salts thereof.

In one embodiment, the invention provides a compound of formula (I):

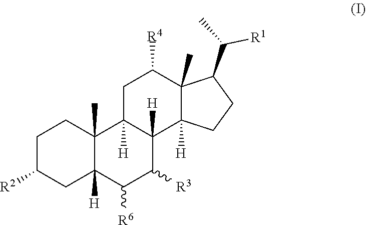

or a salt thereof, wherein:

R¹ is (C₁-C₆)alkyl that is substituted with one or more R⁵, wherein R⁵ is independently selected from the group consisting of halo, cyano, heterocycle, heteroaryl, —OH, —C(═O)NH(C₁-C₆ alkyl)(S(O)₂R^{c}), —C(═O)NH(C₁-C₆ alkyl)(C(═)OR^{c}), —C(O)NR^{a}R^{b}, —OS(O)₃R^{c}, —C(═O)NH(S(O)₂R^{c}), —C(═O)OR^{c}, and —C(═O)(C₁-C₆ alkyl), wherein any heterocycle and heteroaryl is optionally substituted with one or more groups independently selected from the group consisting of halo, carboxy, —OH, cyano, (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy, wherein any (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy is optionally substituted with one or more groups independently selected from the group consisting of —OH and (C₁-C₆)alkoxy;

R² is selected from the group consisting of —OH, —OC(═O)R^{c}, —OS(O)₃H and (C₁-C₆)alkoxy;

R³ is —OS(O)₃R^{e};

R⁴ is selected from the group consisting of H and —OH, where;

R^{a} and R^{b} are independently selected from 1- and (C₁-C₆)alkyl that is optionally substituted with one or more groups independently selected from the group consisting of hydroxy and (C₁-C₆)alkoxy that is optionally substituted with one or more groups independently selected from the group consisting of hydroxy and (C₁-C₆)alkoxy; or R^{a} and R^{b} together with the nitrogen to which they are attached form a 4-6 membered ring heterocycle;

each R^{c} is independently selected from H and (C₁-C₆)alkyl;

R¹ is (C₁-C₆)alkyl;

R⁶ is selected from the group consisting of H and (C₁-C₆)alkyl, or R⁶ and R³ together with the atom to which they are attached form a carbocycle or heterocycle, wherein the carbocycle or heterocycle is optionally Substituted with halo, cyano, heteroaryl, —OH and (C₁-C₆)alkyl.

In one embodiment. R² is not —OC(═O)H.

In one embodiment, R³ is not —OC(═O)H.

In one embodiment, R² is not —OC(═O)H when R³ is —OC(═O)H.

In one embodiment, both R² and R³ are not —OC(═O)H.

In one embodiment, the compounds of formula (I) exclude compounds wherein R² is —OH; R³ is —OC(═O)CH₃; R⁴ is H; and R¹ is (C₁-C₆)alkyl that is substituted with one or more groups independently selected from the group consisting of —OH, heteroaryl, and —C(═O)OR^{c}.

In one embodiment, the invention provides a method for treating a TGR5 mediated condition in an animal (e.g. a human) in need of such treatment comprising administering to the animal a compound of formula (I), or a pharmaceutically acceptable salt thereof.

In one embodiment, the invention provides a method for treating a TGR5 mediated condition in an animal (e.g. a human) comprising administering to the animal an effective TGR5 agonist amount of a compound of formula (I), or a pharmaceutically acceptable salt thereof.

In one embodiment, the invention provides a method for treating a TGR5 mediated condition in an animal (e.g. a human) in need of such treatment comprising administering to the animal an effective TGR5 agonist amount of a compound of formula (I), or a pharmaceutically acceptable salt thereof.

In one embodiment, the invention provides a TGR5 agonizing composition comprising a compound of formula (I), or a pharmaceutically acceptable salt thereof.

In one embodiment, the invention provides an anti-obesity composition comprising a compound of formula (I), or a pharmaceutically acceptable salt thereof.

In one embodiment, the invention provides an anti-inflammatory bowel disease composition comprising a compound of formula (I), or a pharmaceutically acceptable salt thereof.

In one embodiment, the invention provides an anti-diabetic composition comprising a compound of formula (I), or a pharmaceutically acceptable salt thereof.

In cases where compounds are sufficiently basic or acidic, a salt of a compound of formula (I) can be useful as an intermediate for isolating or purifying a compound of formula (I). Additionally, administration of a compound of formula (I) as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of formula (I) can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisns.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula (1) to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula (I) can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The invention will now be illustrated by the following non-limiting Examples.

### EXAMPLES

### Example 1. Synthesis of (3R,5S,7R,8R,9S,10S,13R,14S,17R)-7-methoxy-10,13-dimethyl-17-((R)-4-(5-methyl-1,3,4-oxadiazol-2-yl)butan-2-yl)hexadecahydro-1H cyclopenta[a]phenanthren-3-ol (17)

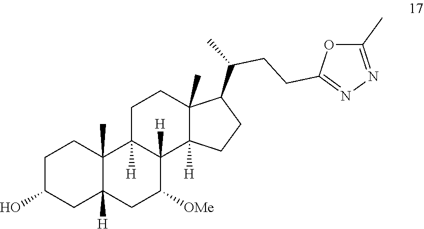

a. Preparation of (R)—N′-acetyl-4-((3R,5R,7R,8R,9S,10S,13R,14S,17R)-3-((tert-butyldimethylsilyl)oxy)-7-methoxy-10,13-dimethylhexadecahydro-1H-cyclopenta[a]-phenanthren-17-yl)pentanehydrazide (15).

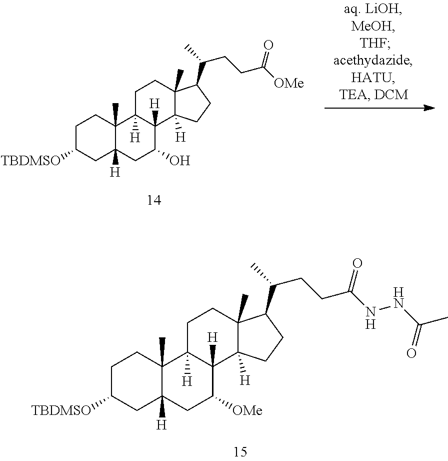

To a solution of compound 14 (Stoltz, K. L. et al. J. Med. Chem. 2017, 60, 3451-3471) (492 mg, 0.919 mmol) in THF (10 mL) and methanol (2 mL) was added 1 aqueous LiOH (2 mL). After stirring at room temperature overnight, the organic solvents were removed by rotary evaporation and the reaction mixture acidified with 1 M aqueous HCl (50 ml) and extracted with EtOAc (2×50 mL). The combined organic layers were dried over Na₂SO₄, filtered, and concentrated to afford a white solid (466.3 mg), which was used in the next step without further purification. To a solution of the acid (466 mg, 0.895 mmol) in DCM (8 mL) was added HIATU (406 mg, 1.07 mmol) and triethylamine (0.105 mL, 0.753 mmol). After stirring for 5 minutes, acethydrazide (94.1 mg, 1.27 mmol) was added. After stirring at room temperature for 3 days, the reaction mixture was diluted with 1 M aqueous HCl (50 ml) and extracted with DCM (2×50 mL). The combined organic layers were dried over Na₂SO₄, filtered, and concentrated. The crude material was purified by flash column chromatography on silica gel (20-100% EtOAc in hexanes as eluent) to obtain a white solid (319.8 mg). As NMR analysis showed this material to be contaminated with likely tetramethyl urea, the solid was dissolved in EtOAc (100 mL) and extracted with water (4×100 mL). The organic layer was then dried over Na₂SO₄, filtered, and concentrated to afford a white solid (264.9 mg, 50% yield from 14). ¹H NMR (400 MHz, CDCl₃) δ: 8.20 (d, J=5.41 Hz, 1H), 8.09 (d, J=5.5 Hz, 1H), 3.47-3.35 (m, 1H),3.23 (s, 3H), 3.20-3.13 (m, 1H), 2.37-2.09 (m, 3H), 2.06 (s, 3H), 1.96-1.70 (m, 6H), 1.64-0.97 (m, 16H), 0.96-0.84 (m, 16H), 0.62 (s, 3H), 0.04 (s, 6H). ¹³C NMR (100 MHz, CDCl₃) δ: 170.5, 166.8, 77.2, 72.9, 55.8, 55.7, 50.2, 42.5, 42.0, 39.6, 39.4, 38.7, 35.49, 35.46, 35.0, 33.6, 31.4, 31.1, 31.0, 28.2, 27.9, 25.9 (3C),23.6, 22.9, 20.8, 20.7, 18.3, 18.2, 11.7, −4.5 (2C). HRMS: m/z calcd. C₃₃H₆₀N₂NaO₄Si (M+Na⁺) 599.4215, found 599.4239.

b. Preparation of 2-((R)-3-((3R,5R,7R,8R,9S,10S,13R,14S,17R)-3-((tert-butyldimethyl-silyl)oxy)-7-methoxy-10,13-dimethylhexadecahydro-1H-cyclopenta[a]phenanthren-17-yl)butyl)-5-methyl-1,3,4-oxadiazole (16)

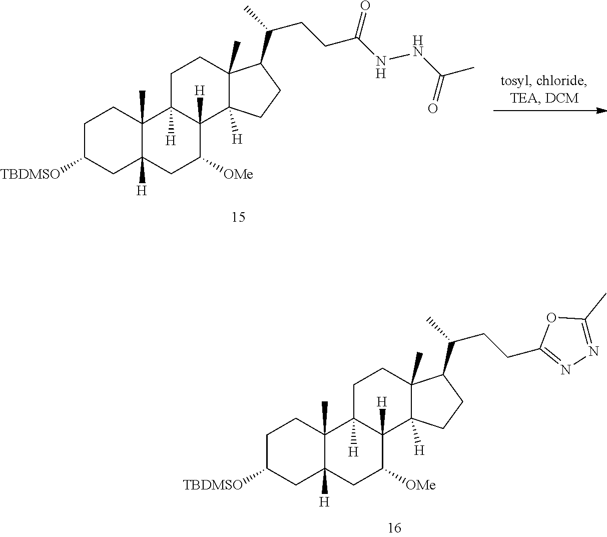

To a solution of compound 15 (252 mg, 0.436 mmol) in DCM (5 mL), was added triethylamine (0.200 mL, 1.43 mmol) followed by tosyl chloride (84.7 mg, 0.444 mmol). More tosyl chloride (43.8 mg, 0.230 mmol) was added the next day and stirring continued for an additional 2 hours. The crude material was purified by flash column chromatography on silica gel (20-100% EtOAc in hexanes as eluent) to furnish compound 16 as a white solid (135.2 mg, 55% yield). ¹H NMR (400 MHz, CDCl₃) δ: 3.43-3.32 (m, 1H), 3.20 (s, 3H), 3.16-3.09 (m, 1H), 2.85-2.74 (m, 1H), 2.73-2.60 (m, 1H), 2.46 (s, 3H), 2.16 (quart, J=12.6 Hz, 1H), 1.93-1.67 (m, 6H), 1.62-0.77 (32H), 0.59 (3H), 0.00 (s, 6H). ¹³C NMR (100 MHz, CDCl₃) δ: 167.6, 163.5, 77.2, 72.9, 55.8, 56.6, 50.2, 42.5, 42.0, 39.6, 39.4, 38.7, 35.5, 35.4, 35.0, 33.7, 32.6, 31.1, 28.2, 27.9, 25.9 (3C), 23.6, 22.9, 22.2, 20.8, 18.3, 18.2, 11.7, 11.0, −4.4 (2C). HRMS: m/z calcd. C₃₃H₅₈N₂NaO₃Si (M+Na⁺) 581.4109, found 581.4103.

c. Preparation of (3R,5S,7R,8R,9S,10S,13R,14S,17R)-7-methoxy-10,13-dimethyl-17-((R)-4-(5-methyl-1,3,4-oxadiazol-2-yl)butan-2-yl)hexadecahydro-1H-cyclopenta[a]phenanthren-3-ol (17).

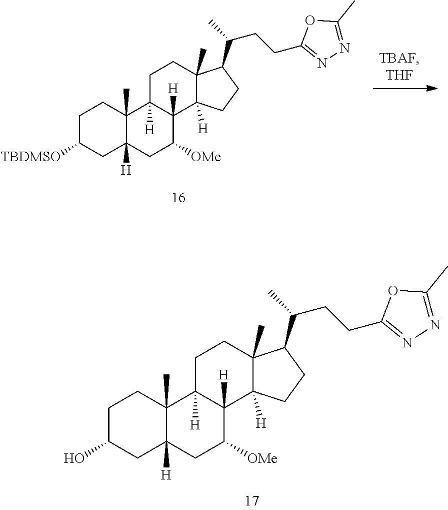

Compound 16 (123.3 mg, 0.221 mmol) was dissolved in 1 M TBAF in THF (3 mL, 3.00 mmol) and stirring continued overnight. The next day, the reaction mixture was diluted with water (100 mL) and extracted with EtOAc (3×100 mL). The combined organic layers were dried over Na₂SO₄, filtered, and concentrated under reduced pressure. The crude material was purified by silica gel column chromatography (30-100% EtOAc in hexanes as eluent) to obtain compound 17 (91.8 mg, 93% yield) as white solid. ¹H NMR (400 MHz, DMSO-D₆) δ: 4.33 (d, J=4.7 Hz, 11-1), 3.26-3.16 (m, 1H), 3.18 (s, 3H), 3.15-3.11 (s, 1H), 2.89-2.77 (m, 1H), 2.76-2.64 (m, 1H), 2.44 (s, 3H), 2.06-1.87 (m, 2H), 1.87-0.98 (m, 21H), 0.96-0.87 (m, 1H), 0.94 (d, J=5.8 Hz, 3H), 0.86 (s, 3H), 0.61 (s, 3H). ¹³C NMR (100 MHz, CDCl₃) δ: 167.6, 163.5, 77.5, 72.1, 55.9, 55.6, 50.3, 42.5, 41.9, 39.6, 39.4, 38.5, 35.4, 35.3, 35.0, 33.8, 32.6, 30.9, 28.2, 27.8, 23.6, 22.9, 22.2, 20.8, 18.3, 1.7, 1.0. LC/MS (ESI): m/z calcd. C₂₇H₄₅N₂O₄ (M+H⁺) 445.3, found 445.3.

### Example 2. Synthesis of (R)-4-((3R,5S,7R,8R,9S,10S,13R,14S,17R)-3-hydroxy-7-methoxy-10,13-dimethylhexadecahydro-1H-cyclopenta[a]phenanthren-17-yl)-1-(pyrrolidin-1-yl)pentan-1-one (12)

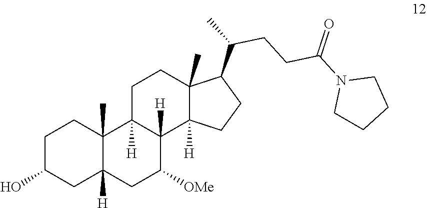

a. Preparation of (R)-4-((3R,5R,7R,8R,9S,10S,13R,14S,17R)-3-((tert-butyldimethyl-silyl)oxy)-7-hydroxy-10,13-dimethylhexadecahydro-1-cyclopenta[a]phenanthren-17-yl)-1-(pyrrolidin-1-yl)pentan-1-one (10)

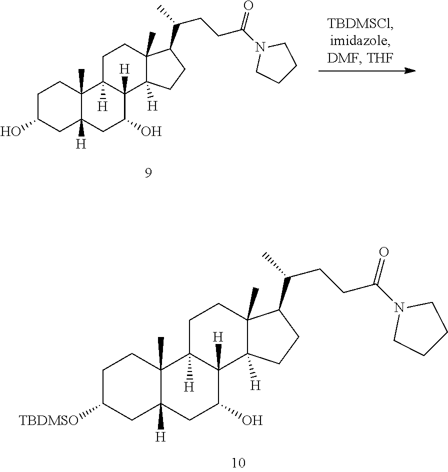

To a solution of compound 9 (782 mg, 1.75 mmol) (Stoltz, K. L. et al. J. Med. Chem. 2017, 60, 3451-3471) and imidazole (1.45 g, 21.3 mmol) in DMF (8 mL), THF (3 mL), and pyridine (1 mL) was added TBDMSCl (352 mg, 2.33 mmol). After stirring overnight, TLC analysis showed remaining starting material, so more TBDMSCl (124 mg, 0.82 mmol) was added. After stirring for an additional 90 minutes, the reaction mixture was diluted with 1 M HCl (150 mL) and extracted with EtOAc (2×200 mL) The combined organic layers were dried over Na₂SO₄, filtered, and concentrated under reduced pressure. The crude material was purified by silica gel column chromatography (40-100% EtOAc in hexanes as eluent) to obtain 10 (884 mg, 90% yield) as white solid. ¹H NMR (400 MHz, CDCl₃) δ: 3.86-3.79 (m, 1H), 3.49-3.37 (m, 5H), 2.35-2.09 (m, 3H), 2.01-1.74 (m, 10H), 1.62-1.07 (m, 17H) 0.99-0.92 (m, 1H), 0.94 (d, J=6.2 Hz, 3H), 0.87 (s, 12H), 0.65 (s, 3H), 0.04 (s, 6H). ¹³C NMR (100 MHz, CDCl₃) δ: 172.2, 72.9, 68.6, 55.8, 50.5, 46.6, 45.6, 42.7, 41.6, 40.1, 39.6, 39.5, 35.6, 35.5, 35.1, 34.6, 32.8, 31.5, 31.1, 30.9, 28.2, 26.2, 26.0 (3C),24.4, 23.8, 22.8, 20.6, 18.5, 19.3, 11.8, −4.5, −4.6. HRMS: m/z calcd. C₃₄H₆₁NNaO₃Si (M+Na⁺) 582.4313, found 582.4309.

b. Preparation of (R)-4-((3R,5S 7R,8R,9S,10S,13R,14S,17R)-3-hydroxy-7-methoxy-10,13-dimethylhexadecahydro-1H-cyclopenta[a]phenanthren-17-yl)-1-(pyrrolidin-1-yl)pentan-1-one (12).

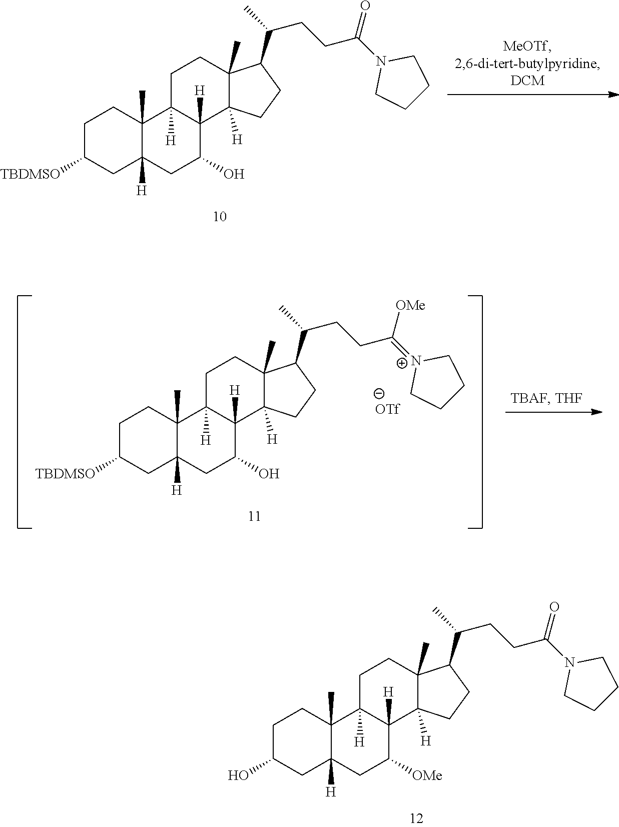

To a solution of compound 10 (95.6 mg, 0.171 mmol) in DCM (1 mL) and 2,6-di-tert-butylpyridine (0.180 mL, 0.713 mmol) was added methyl trifluoromethanesulfonate (0.080 mL, 0.707 mmol). After stirring overnight, the reaction mixture was diluted with water (10 mL) and extracted with DCM (10 mL). The organic layer was dried over Na₂SO₄, filtered, and concentrated under reduced pressure. To this crude material was added a solution of 1 M TBAF in THF (2 mL). After stirring at room temperature for 2 days, the mixture was concentrated under reduced pressure, diluted with water (10 mL) and extracted with EtOAc (2×10 mL). The combined organic layers were dried over Na₂SO₄, filtered, and concentrated under reduced pressure. The crude material was purified by flash column chromatography on silica gel (100% EtOAc as eluent) to obtain 12 as a white solid (8.8 ng, 9% yield). H NMR (400 MHz, CDCl₃) δ: 3.44-3.29 (m, 5H), 3.18 (s, 3H), 3.15-3.07 (s, 1H), 2.30-2.17 (m, 1H), 2.16-2.00 (m, 2H), 1.95-1.66 (m, 10H), 1.66-0.94 (m, 17H), 0.95-0.80 (m, 1H), 0.87 (d, J=6.4 Hz, 3H), 0.83 (s, 3H), 0.57 (s, 3H). ¹³C NMR (100 MHz, CDCl₃) δ: 172.3, 77.6, 72.1, 55.9, 55.8, 50.3, 46.6, 45.6, 42.5, 42.0, 39.6, 39.4, 38.6, 35.5, 35.3, 35.0, 33.8, 31.5, 30.92, 30.89, 28.2, 27.8, 26.2, 24.4, 237, 22.9, 20.9, 18.5, 11.7. LC/MS (ESI): m/z calcd. C₂₉H₅₀NO₃ (M+H⁺) 460.4, found 460.4.

### Example 3. Synthesis of (R)-4-((3R,5S,7R,8R,9S,10S,13R,14S,17R)-3-hydroxy-7-methoxy-10,13-dimethylhexadecahydro-1H-cyclopenta[a]phenanthren-17-yl)pentanoic acid

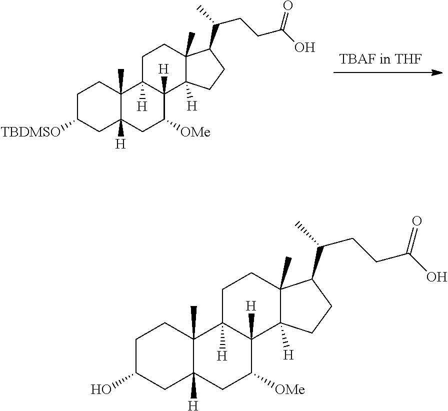

(R)-4-((3R,5S,7R,8R,9S,10S,13R,14S,17R)-3-((tert-butyldimethylsilyl)oxy)-7-methoxy-10,13-dimethylhexadecahydro-1H-cyclopenta[a]phenanthren-17-yl)pentanoic acid (581 mg, 1.12 mmol) was dissolved in 1 M TBAF in THF (6 mL). After stirring at rt for 5 days, the solution was diluted with 1 M HCl (100 mL) and extracted with DCM (2×100 mL). The combined organic layers were dried over Na₂SO₄, filtered, and concentrated. The crude material was purified by flash column chromatography on silica gel (80-100% EtOAc in hexanes as eluent) to obtain the title compound (328 mg, 82% yield) as a white solid. ¹H NMR (400 MHz, DMSO-D₆) δ 11.92 (bs, 1H), 4.34 (bs, 1H), 3.18 (s, 3H), 3.25-3.10 (m, 2H), 2.30-2.18 (m, 1H), 2.17-0.97 (n, 24H), 0.95-0.83 (m, 1H), 0.88 (d, J=6.5 Hz, 3H), 0.86 (s, 3H), 0.62 (s, 3H). δ ¹³C NMR (100 MHz, DMSO-D₆) δ 174.9, 76.8, 70.1, 55.5, 55.3, 49.9, 41.9, 41.3, 39.3, 38.9, 38.1, 35.1, 34.9, 34.6, 33.3, 30.7, 30.6, 30.5, 27.6, 27.3, 23.2, 22.7, 20.4, 18.1, 11.5. LC/MS (ESI): m/z calcd. C₂₅H₄₁O₄ (M−H) 405.3, found 405.3.

### Example 4. Synthesis of (R)-4-((3R,5S,7R,8R,9S,10S,13R,14S,17R)-3-hydroxy-7-methoxy-10,13-dimethylhexadecahydro-1H-cyclopenta[a]phenanthren-17-yl)-N-(2-(2-methoxyethoxy)ethyl)pentanamide

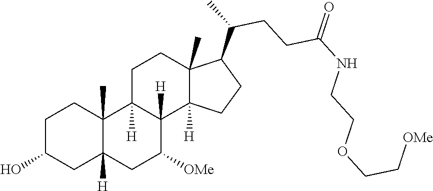

a. Preparation of (R)-4-((3R,5R,7R,8R,9S,10S,13R,14S,17R)-3-((tert-butyldimethyl-silyl)oxy)-7-methoxy-10,13-dimethylhexadecahydro-1H-cyclopenta[a]phenanthren-17-yl)-N-(2-(2-methoxyethoxy)ethyl)pentanamide.

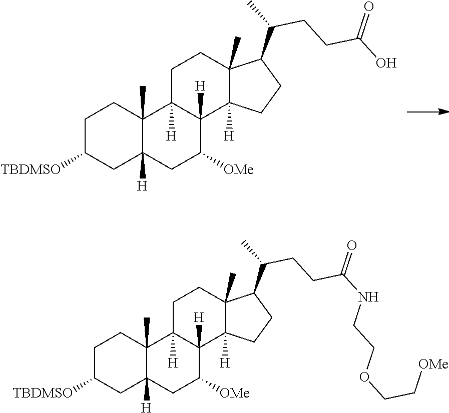

To a solution of (R)-4-((3R,5R,7R,8R,9S,10S,13R,4S,17R)-3-((tert-butyldimethyl-silyl)oxy)-7-methoxy-10,13-dimethylhexadecahydro-H-cyclopenta[a]phenanthren-17-yl)pentanoic acid (114.5 mg, 0.220 mmol) in DCM (4 mL) was added DIEA (0.075 mL) and HATU (87.5 mg, 0.230 mmol). After stirring for 12 minutes, 2-(2-methoxyethoxy)ethanamine (0.030 mL, 0.241 mmol) was added by syringe. The next day, the crude material was purified by flash column chromatography on silica gel (35-100% EtOAc in hexanes as eluent). The resulting material was dissolved in EtOAc (75 mL) and extracted with water (4×75 mL) to obtain the title compound (131.7 mg, 96% yield) as a colorless oil.

b. Preparation of (R)-4-((3R,5S,7R,8R,9S,10S,13R,14S,17R)-3-hydroxy-7-methoxy-10,13-dimethylhexadecahydro-1H-cyclopenta[a]phenanthren-17-yl)-N-(2-(2-methoxyethoxy)ethyl)pentanamide.

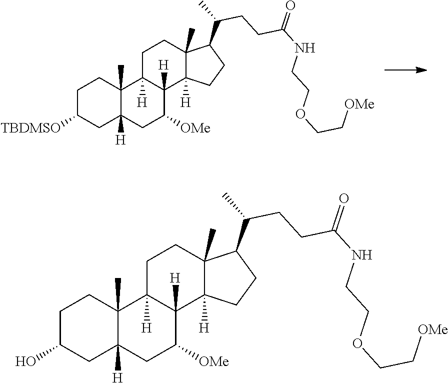

(R)-4-((3R,5R,7R,8R,9S,10S,13R,14S,17R)-3-((tert-butyldimethylsilyl)oxy)-7-methoxy-10,13-dimethylhexadecahydro-1H-cyclopenta[a]phenanthren-17-yl)-N-(2-(2-methoxyethoxy)ethyl)pentanamide (124.1 mg, 0.200 mmol) was dissolved in 1 M TBAF in T-F (2.5 mL). After stirring at room temperature for 5 days, the solution was diluted with water (60 mL) and extracted with EtOAc (60 mL). The organic layer was dried over Na₂SO₄, filtered, and concentrated. The crude material was purified by flash column chromatography on silica gel (0-7% MeOH in EtOAc as eluent) to obtain the title compound (60.9 mg, 60% yield) as a clear, colorless oil. ¹³C NMR (100 MHz, CDCl₃) δ 173.7, 77.5, 72.1, 71.9, 70.2, 70.0, 59.1, 55.9, 55.8, 50.3, 42.5, 41.9, 39.6, 39.4, 39.1, 38.5, 35.5, 35.3, 35.0, 33.7, 33.5, 31.8, 30.9, 28.2, 27.8, 23.7, 22.9, 20.8, 18.4, 11.7. LC/MS (EST): m/calcd. C₃₀H₅₄NO₅ (M+H⁺) 508.4, found 508.4.

### Example 5. Synthesis of (3R,5S,7R,8R,9S,10S,13R,14S,17R)-7-methoxy-17-((R)-4-(5-(methoxymethyl)-1,3,4-oxadiazol-2-yl)butan-2-yl)-10,13-dimethylhexadecahydro-1-cyclopenta[a]phenanthren-3-ol

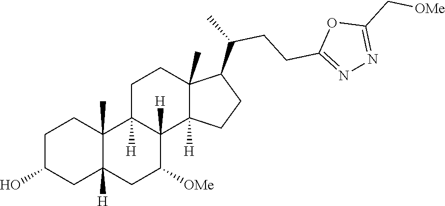

a. Preparation of (R)-4-(3R,5R,7R,8R,9S,10S,13R,14S,17R)-3-((tert-butyldimethyl-silyl)oxy)-7-methoxy-10,13-dimethylhexadecahydro-1H-cyclopenta[a]phenanthren-17-yl)-N-(2-methoxy acetyl)pentanehydrazide.

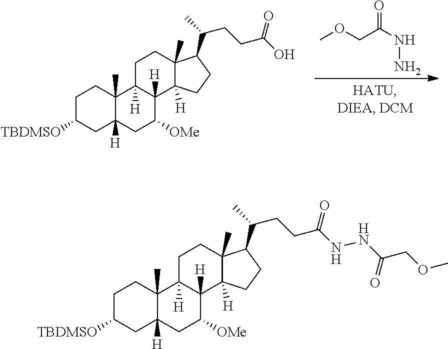

To a solution of (R)-4-((3R,5R,7R,8R,9S,10S,13R,14S,17R)-3-((tert-butyldimethyl-silyl)oxy)-7-methoxy-10,13-dimethylhexadecahydro-1H-cyclopenta[a]phenanthren-17-yl)pentanoic acid (163.3 mg, 0.314 mmol) in DCM (6 mL) was added DIEA (0.100 mL) and HATU (128 mg, 0.337 mmol). After stirring for 10 minutes, 2-methoxyacetohydrazide (37.2 mg, 0.357 mmol) was added. After stirring for three days, the crude material was purified by flash column chromatography on silica gel (40-100% EtOAc in hexanes as eluent). The resulting material was dissolved in EtOAc (75 mL) and extracted with water (3×75 mL) to obtain the title compound (177 mg, 93% yield) as a white solid.

b. Preparation of 2-((R)-3-((3R,5R,7R,8R,9S,10S,13R,14S,17R)-3-((tert-butyl-dimethylsilyl)oxy)-7-methoxy-10,13-dimethylhexadecahydro-1H-cyclopenta[a]phenanthren-17-yl)butyl)-5-(methoxymethyl)-1,3,4-oxadiazole.

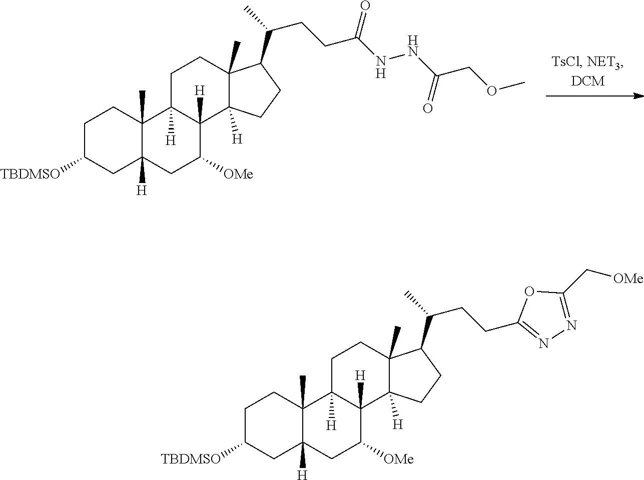

To a solution of (R)-4-((3R,5R,7R,8R,9S,10S,13R,14S,17R)-3-((tert-butyl-dimethylsilyl)oxy)-7-methoxy-10,13-dimethylhexadecahydro-1H-cyclopenta[a]phenanthren-17-yl)-N′-(2-methoxyacetyl)pentanehydrazide (168.8 mg, 0.278 mmol) in DCM (3 mL) and triethylamine (0.2 mL) was added 4-toluenesulfonyl chloride (59 mg, 0.309 mmol). After stirring overnight, the crude material was purified by flash column chromatography on silica gel (20-100% EtOAc in hexanes as eluent) to obtain the title compound (106.7 mg, 65% yield) as a clear, colorless oil.

c. Preparation of (3R,5S,7R,8R,9S,10S,13R,14S,17R)-7-methoxy-17-((R)-4-(5-(methoxymethyl)-1,3,4-oxadiazol-2-yl)butan-2-yl)-10,13-dimethylhexadecahydro-1H-cyclopenta[a]phenanthren-3-ol.

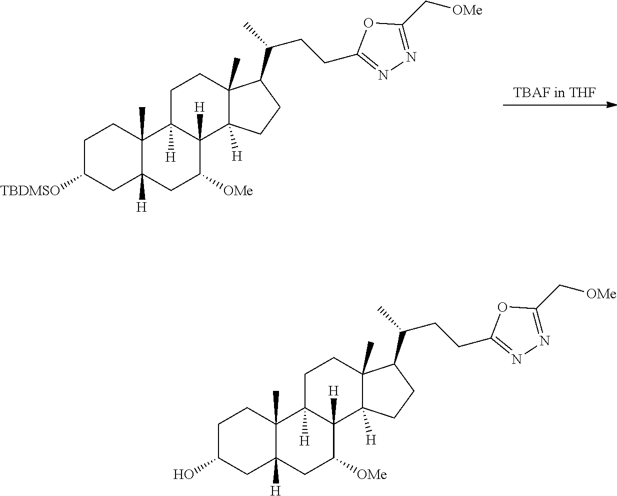

2-((R)-3-((3R,5R,7R,8R,9S,10S,13R,14S,17R)-3-((tert-butyldimethylsilyl)oxy)-7-methoxy-10,13-dimethylhexadecahydro-H-cyclopenta[a]phenanthren-17-yl)butyl)-5-(methoxymethyl)-1,3,4-oxadiazole (100 mg, 0.170 mmol) was dissolved in TI-IF (2 mL) and 1 M TBAF in THF (1.0 mL). After stirring at room temperature for overnight, a TLC showed some starting material remaining. Additional 1 M TBAF in THF (2.0 mL) was added and the solution heated to 50° C. for three hours. The solution was then diluted with water (75 mL) and extracted with EtOAc (2×75 mL). The combined organic layer was dried over Na₂SO₄, filtered, and concentrated. The crude material was purified by flash column chromatography on silica gel (35-100% EtOAc in hexanes as eluent) to obtain the title compound (74.7 mg, 93% yield) as a white solid. ¹H NMR (400 MHz, CDCl₃) δ 4.62 (s, 2H), 3.49-3.39 (n, 1), 3.45 (s, 3H), 3.25 (s, 3H), 3.21-3.17 (m, 1H), 2.95-2.85 (m, 1H), 2.82-2.72 (m, 1H), 2.22-2.10 (m, 1H), 1.99-1.74 (n. 6H), 1.71-0.88 (m, 17H), 0.99 (d, J=6.2 Hz, 311), 0.91 (s, 311), 0.65 (s, 311). LC/MS (ESI): m/z calcd. C₂₈H₄₇N₂O₄ (M+H⁺) 475.3, found 475.4.

### Example 6. Synthesis of (R)-4-((3R,5S,7R,8R,9S,10S,13R,14S,17R)-3-hydroxy-7-methoxy-10,13-dimethylhexadecahydro-1H-cyclopenta[a]phenanthren-17-yl)-N-((2S,3R,4R,5R)-2,3,4,5,6-pentahydroxyhexyl)pentanamide

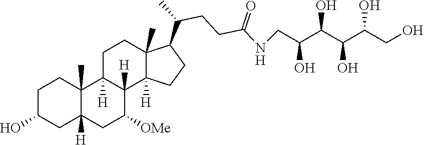

a. Preparation of (R)-4-((3R,5R,7R,8R,9S,10S,13R,14S,17R)-3-((tert-butyl-dimethylsilyl)oxy)-7-methoxy-10,13-dimethylhexadecahydro-1H-cyclopenta[a]-phenanthren-17-yl)-N-((2S,3R,4R,5R)-2,3,4,5,6-pentahydroxyhexyl)pentanamide.

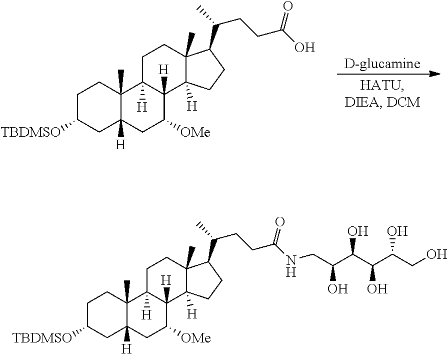

To a solution of (R)-4-((3R,5R,7R,8R,9S,10S,13R,14S,17R)-3-((tert-butyl-dimethylsilyl)oxy)-7-methoxy-10,13-dimethylhexadecahydro-1H-cyclopenta[a]-phenanthren-17-yl)pentanoic acid (100 mg, 0.192 mmol) in DCM (4 mL) was added DIEA (0.075 mL) and HATU (77 mg, 0.203 mmol). After stirring for 10 minutes, D-glucamine (42 ng, 0.232 mmol) was added. After stirring overnight, the reaction mixture was diluted with water (50 mL) and extracted with DCM (2×50 nL). The combined organic layer was dried over Na₂SO₄, filtered, and concentrated. The crude material was purified by flash column chromatography on silica gel (0-10% MeOH in EtOAc as eluent) to obtain the title compound (49.7 mg, 38% yield).

b. Preparation of (R)-4-((3R,5S,7R,8R,9S,10S,13R,14S,17R)-3-hydroxy-7-methoxy-10,13-dimethylhexadecahydro-1H-cyclopenta[a]phenanthren-17-yl)-N-((2S, 3R,4R,5R)-2,3,4,5,6-pentahydroxyhexyl)pentanamide.

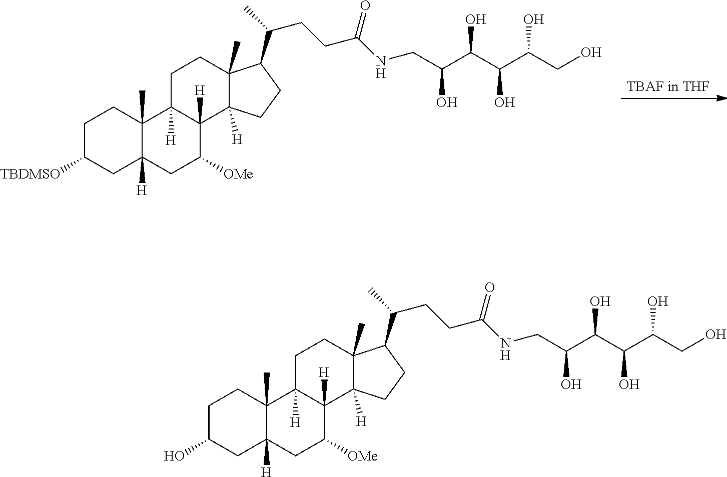

(R)-4-((3R,5R,7R,8R,9S,10S,13R,14S,17R)-3-((tert-butyldimethylsilyl)oxy)-7-methoxy-10,13-dimethylhexadecahydro-1H-cyclopenta[a]phenanthren-17-yl)-N-((2S,3R,4R,5R)-2,3,4,5,6-pentahydroxyhexyl)pentanamide (49.5 mg, 0.095 mmol) was dissolved in 1 M TBAF in THF (2.0 mL). After stirring at room temperature for 4 days, the reaction mixture was concentrated and the resulting residue was purified by liquid chromatography (10% CH₃CN/water to 100% CH₃CN, C18 column) to yield, after lyophilization, the title compound (31.9 mg, 59%) as a white solid. L C/MS (ESI): m/z calcd. C₃₁H₅₆NO₈ (M+H⁺) 570.4, found 570.4.

### Example 7. Synthesis of (R)-4-((3R,5S,7R,8R,9S,10S,13R,14S,17R)-3-hydroxy-7-methoxy-10,13-dimethylhexadecahydro-1H-cyclopenta[a]phenanthren-17-yl)-N-methyl-N-((2S,3R,4R,5R)-2,3,4,5,6-pentahydroxyhexyl)pentanamide

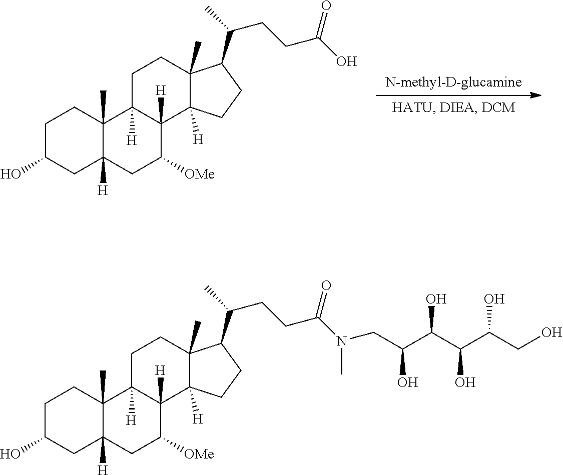

To a solution of (R)-4-((3R,5S,7R,8R,9S,10S,13R,14S,17R)-3-hydroxy-7-methoxy-10,13-dimethylhexadecahydro-1H-cyclopenta[a]phenanthren-7-yl)pentanoic acid (58.6 mg, 0.144 mmol) in DMF (1.5 mL) was added DIEA (0.050 mL) and HATU (55.2 mg, 0.145 mmol). After stirring for 5 minutes, N-methyl-D-glucamine (31.1 mg, 0.159 mmol) was added. After stirring overnight, the reaction mixture was purified by liquid chromatography (10% CH₃CN/water to 100% CH₃CN, C18 column) to yield, after lyophilization, the title compound (70.3 mg, 84%) as a white solid. LC/MS (ESI): m/z calcd. CH₃₂H₅₈NO₈ (M+H⁺) 584.4, found 585.5.

TGR5 agonist activity was evaluated by assaying cAMP production in a recombinant cell line expressing human TGR5 receptor as described in Example 8.

### Example 8. TGR5 cAMP Assay

TGR5 agonist activity was evaluated by assaying cAMP production in a recombinant cell line expressing human TGR5 receptor. Data for representative compounds is provided in the following table.

| Example | EC₅₀ (nM) |
|---|---|
| 1 | 5.6 |
| 2 | 10.5 |
| 3 | 74.9 |
| 4 | 49.5 |
| 5 | 22.6 |
| 6 | 27.7 |
| 7 | 60.1 |

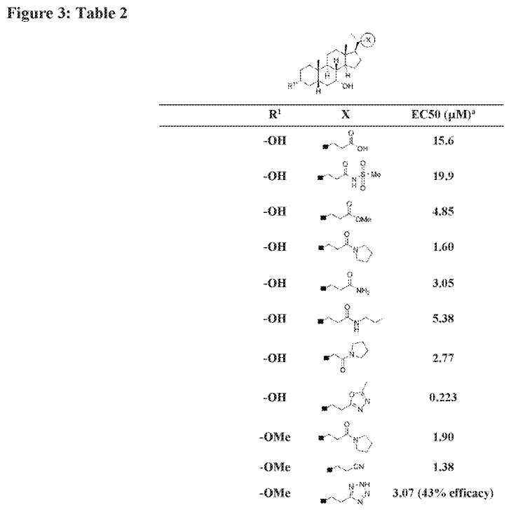

A comparison of this data and the data presented in FIG. 2 and FIG. 3 demonstrates that potency improved when the 7-hydroxy group was replaced with a 7-methoxy group.

### Example 9

The following illustrate representative pharmaceutical dosage forms, containing a compound of formula (1) (‘Compound X’), for therapeutic or prophylactic use in humans.

| (i) Tablet 1 | mg/tablet |
|---|---|
| Compound X= | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Compound X= | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X= | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| Compound X = (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/ml) | mg/ml |
|---|---|
| Compound X = (free acid form) | 10.0 |
| Monobasic sodium phosphate | 0.3 |
| Dibasic sodium phosphate | 1.1 |
| Polyethylene glycol 400 | 200.0 |
| 1.0N Sodium hydroxide solution | q.s. |
| (pH adjustment to 7.0-7.5) | |
| Water for injection | q.s. ad 1 mL |

| (vi) Aerosol | mg/can |
|---|---|
| Compound X= | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications (including A. Nakhi, et al., J. Med. Chem. 2019, 62, 6824-2830), patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed:

1. A method for treating a TGR5 mediated condition in an animal comprising administering to the animal a compound of formula (1): 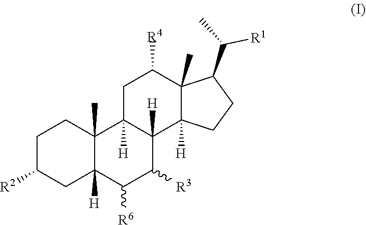
or a pharmaceutically acceptable salt thereof, wherein:
R¹ is (C₁-C₆)alkyl that is substituted with one or more R⁵, wherein R⁵ is independently selected from the group consisting of halo, cyano, heterocycle, heteroaryl, —OH, —C(═O)NH(C₁-C₆ alkyl)(S(O)₂R^{c}), —C(═O)NH(C₁-C₆ alkyl)(C(O)OR^{c}), —C(═O)NR^{a}R^{b}, —OS(O)₃R^{c}, —C(═O)NH(S(O)₂R^{c}), —C(═O)OR^{c}, and —C(═O)(C₁-C₆ alkyl), wherein any heterocycle and heteroaryl is optionally substituted with one or more groups independently selected from the group consisting of halo, carboxy, —OH, cyano, (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy, wherein any (C₁-C₆)alkyl, (C₃-C₆)cycloalky, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy is optionally substituted with one or more groups independently selected from the group consisting of —OH and (C₁-C₆)alkoxy;
R² is selected from the group consisting of —OH, —OC(═O)R^{c}, —OS(O)₃H and (C₁-C₆)alkoxy;
R³ is selected from the group consisting of —OC(═O)R^{d}, (C₁-C₆)alkyl, —OS(O)₃R^{c} and (C₁-C₆)alkoxy;
R⁴ is selected from the group consisting of H and —OH, where;
R^{a} and R^{b} are independently selected from H and (C₁-C₆)alkyl that is optionally substituted with one or more groups independently selected from the group consisting of hydroxy and (C₁-C₆)alkoxy that is optionally substituted with one or more groups independently selected from the group consisting of hydroxy and (C₁-C₆)alkoxy; or R^{a} and R^{b} together with the nitrogen to which they are attached form a 4-6 membered ring heterocycle;
each R^{c} is independently selected from H and (C₁-C₆)alkyl;
R^{d} is H or (C₂-C₆)alkyl; or when R² is —OH, R^{d} is H or (C₁-C₆)alkyl;
R^{e} is (C₁-C₆)alkyl; and
R⁶ is selected from the group consisting of H and (C₁-C₆)alkyl, or R⁶ and R³ together with the atom to which they are attached form a carbocycle or heterocycle, wherein the carbocycle or heterocycle is optionally substituted with halo, cyano, heteroaryl, —OH and (C₁-C₆)alkyl.

2. The method of claim 1, wherein:
R¹ is (C₁-C₆)alkyl that is substituted with one or more R⁵, wherein R⁵ is independently selected from the group consisting of halo, cyano, heterocycle, heteroaryl, —OH, —C(═O)NH—(C₁-C₆ alkyl)(S(O)₂R^{c}), —C(═O)NH(C₁-C₆ alkyl)(C(═O)OR^{c}), —C(═O)NR^{a}R^{b}, —OS(O)₃R^{c}, —C(═O)NH(S(O)₂R^{c}), —C(═O)OR^{c}, and —C(═O)(C₁-C₆ alkyl), wherein any heterocycle and heteroaryl is optionally substituted with one or more groups independently selected from the group consisting of halo, carboxy, —OH, cyano, (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy, wherein any (C₁-C₆)alkyl, (C₃-C₆)cycloalkyl, (C₁-C₆)alkoxy (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy is optionally substituted with one or more groups independently selected from the group consisting of —OH and (C₁-C₆)alkoxy;
R² is selected from the group consisting of —OH, —OC(═O)R^{c}, —OS(O)₃H and (C₁-C₆)alkoxy;
R³ is selected from the group consisting of —OC(═O)R^{d}, (C₁-C₆)alkyl, —OS(O)₃R^{e} and (C₁-C₆)alkoxy;
R⁴ is selected from the group consisting of H and —OH, where;
R^{a} and R^{b} are independently selected from H and (C₁-C₆)alkyl that is optionally substituted with one or more groups independently selected from the group consisting of hydroxy and (C₁-C₆)alkoxy that is optionally substituted with one or more groups independently selected from the group consisting of hydroxy and (C₁-C₆)alkoxy; or R^{a} and R^{b} together with the nitrogen to which they are attached form a 4-6 membered ring heterocycle;
each R^{c} is independently selected from H and (C₁-C₆)alkyl;
R^{d} is (C₂-C₆)alkyl;
R^{e} is (C₁-C₆)alkyl; and
R⁶ is selected from the group consisting of H and (C₁-C₆)alkyl, or R⁶ and R³ together with the atom to which they are attached form a carbocycle or heterocycle, wherein the carbocycle or heterocycle is optionally substituted with halo, cyano, heteroaryl, —OH and (C₁-C₆)alkyl.

3. The method of claim 1, wherein R¹ is (C₂)alkyl that is substituted with one or more R⁵, wherein R⁵ is independently selected from the group consisting of halo, cyano, heterocycle, heteroaryl, —OH, —C(═O)NH(C₁-C₆ alkyl)(S(O)₂R^{c}), —C(═O)NH(C₁-C₆ alkyl)(C(═O)OR^{c}), —C(═O)NR^{a}R^{b}, —OS(O)₃R^{c}, —C(═O)NH(S(O)₂R^{c}), —C(═O)OR^{c}, and —C(═O)(C₁-C₆ alkyl), wherein any heterocycle and heteroaryl is optionally substituted with one or more groups independently selected from the group consisting of halo, carboxy, —OH, cyano, (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy, wherein any (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy is optionally substituted with one or more groups independently selected from the group consisting of —OH and (C₁-C₆)alkoxy.

4. The method of claim 1, wherein R¹ is

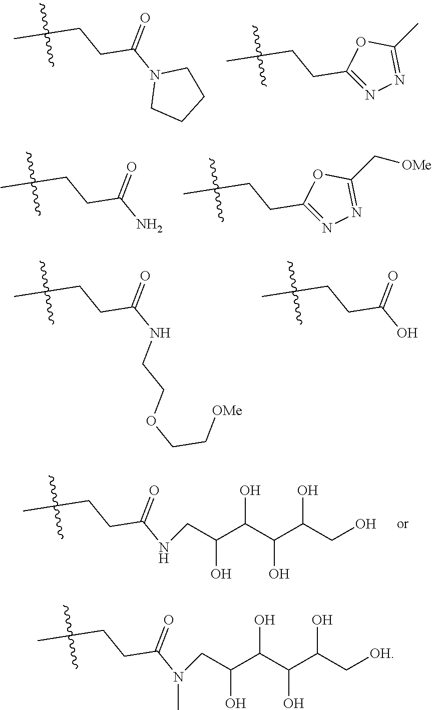

5. The method of claim 1, wherein R¹ is

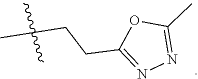 [SMILES: *CCc1nnc(C)o1]

6. The method of claim 1, wherein R² is —OH.

7. The method of claim 1, wherein R³ is —OMe or —OC(═O)H.

8. The method of claim 1, wherein R³ is —OMe.

9. The method of claim 1, wherein R⁴ is H.

10. The method of claim 1, wherein R⁶ is H.

11. The method of claim 1, wherein the compound or salt is selected from: 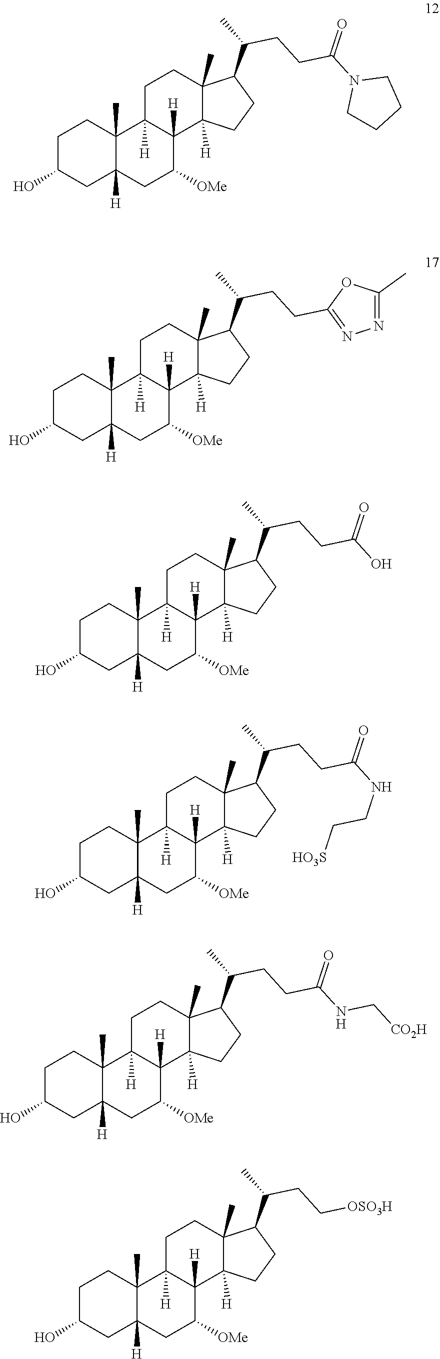 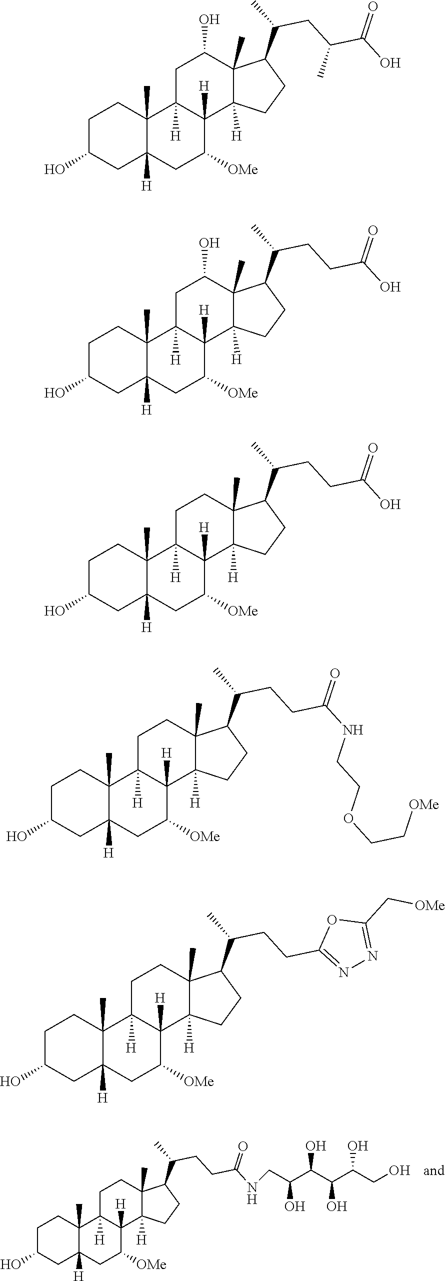 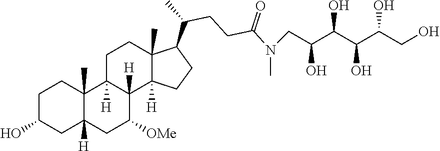
and salts thereof.

12. The method of claim 1, wherein TGR5 mediated condition is diabetes.

13. The method of claim 1, wherein TGR5 mediated condition is obesity.

14. The method of claim 1, wherein TGR5 mediated condition is inflammatory bowel disease.

15. A compound of formula (I): 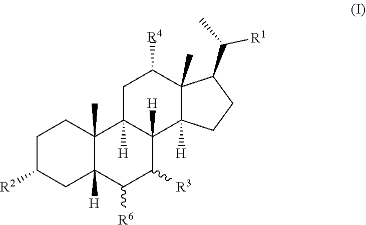
or a salt thereof, wherein:
R¹ is (C₁-C₆)alkyl that is substituted with one or more R⁵, wherein R⁵ is independently selected from the group consisting of halo, cyano, heterocycle, heteroaryl, —OH, —C(═O)NH(C₁-C₆ alkyl)(S(O)₂R^{c}), —C(═O)NH(C₁-C₆ alkyl)(C(═O)OR^{c}), —C(═O)NR^{a}R^{b}, —OS(O)₃R^{c}, —C(═O)NH(S(O)₂R^{c}), —C(═O)OR^{c}, and —C(═O)(C₁-C₆ alkyl), wherein any heterocycle and heteroaryl is optionally substituted with one or more groups independently selected from the group consisting of halo, carboxy, —OH, cyano, (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy, wherein any (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy is optionally substituted with one or more groups independently selected from the group consisting of —OH and (C₁-C₆)alkoxy;
R² is selected from the group consisting of —OH, —OC(═O)R^{c}, —OS(O)₃H and (C₁-C₆)alkoxy;
R³ is selected from the group consisting of —OC(═O)R^{d}, (C₁-C₆)alkyl, —OS(O)₃R^{c} and (C₁-C₆)alkoxy;
R⁴ is selected from the group consisting of H and —OH, where;
R^{a} and R^{b} are independently selected from H and (C₁-C₆)alkyl that is optionally substituted with one or more groups independently selected from the group consisting of hydroxy and (C₁-C₆)alkoxy that is optionally substituted with one or more groups independently selected from the group consisting of hydroxy and (C₁-C₆)alkoxy; or R^{a} and R^{b} together with the nitrogen to which they are attached form a 4-6 membered ring heterocycle;
each R^{c} is independently selected from H and (C₁-C₆)alkyl;
R^{d} is H or (C₂-C₆)alkyl; or when R² is —OH, R^{d} is H or (C₁-C₆)alkyl;
R^{e} is (C₁-C₆)alkyl; and
R⁶ is selected from the group consisting of H and (C₁-C₆)alkyl, or R⁶ and R³ together with the atom to which they are attached form a carbocycle or heterocycle, wherein the carbocycle or heterocycle is optionally substituted with halo cyano, heteroaryl, —OH and (C₁-C₆)alkyl; and
provided that the compound or salt is not selected from the group consisting of: 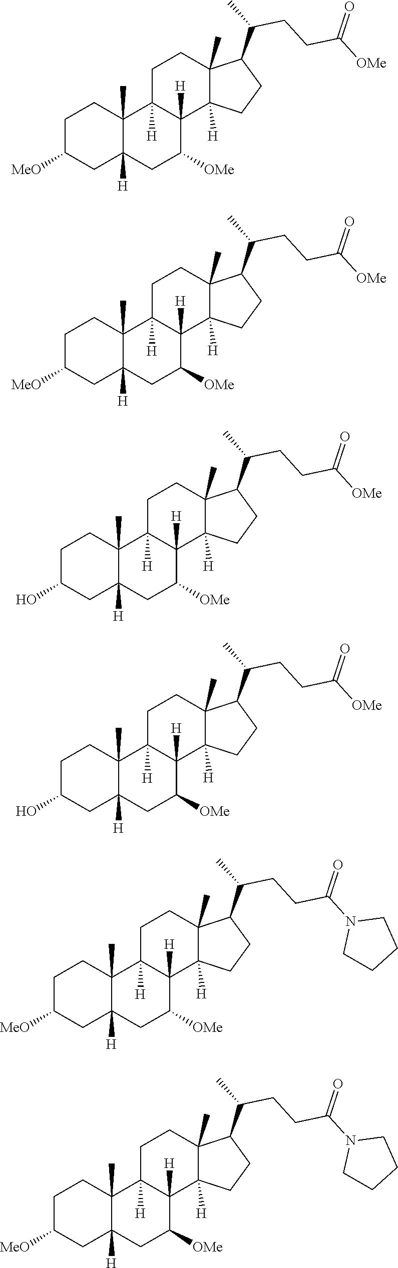 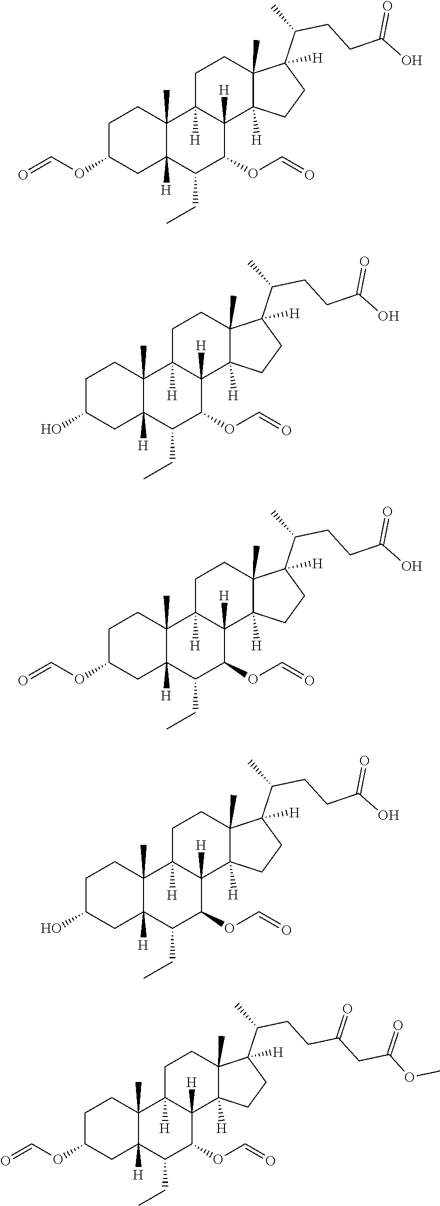 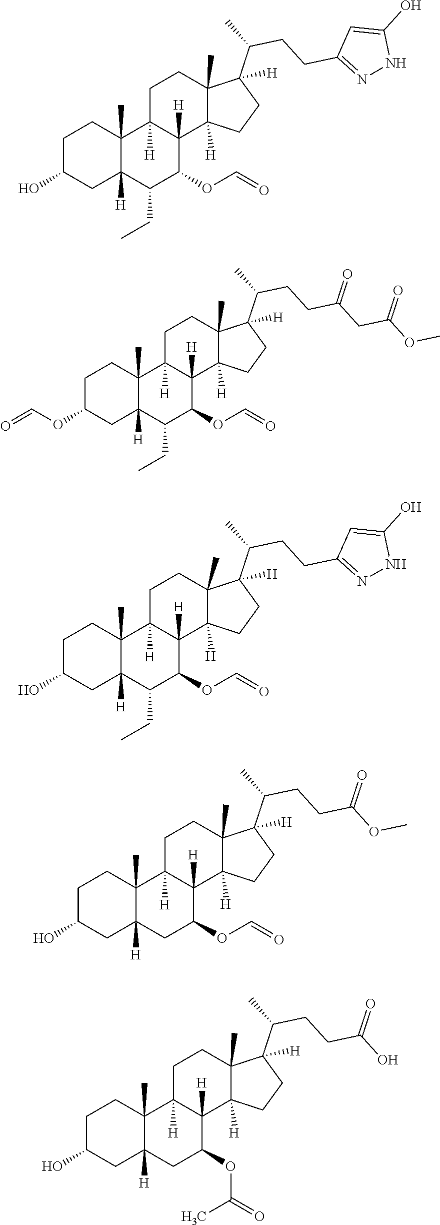 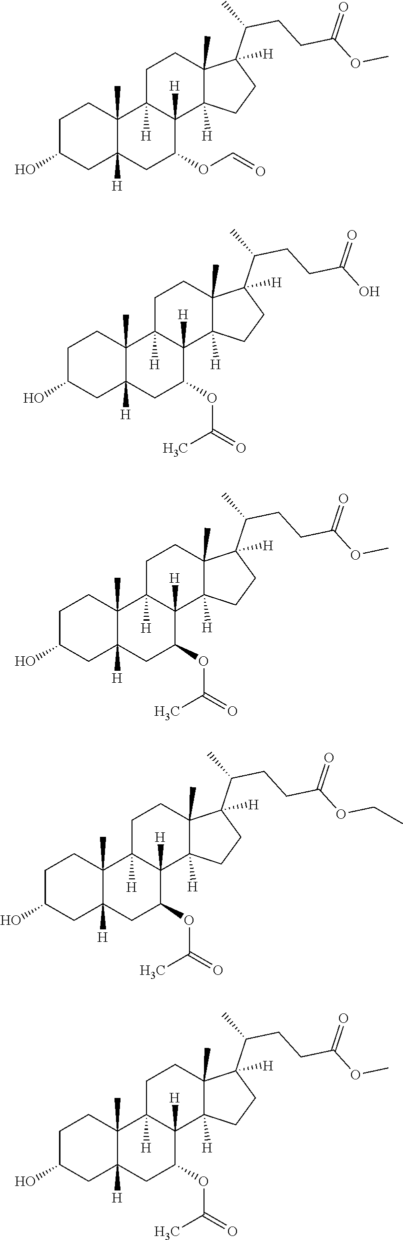 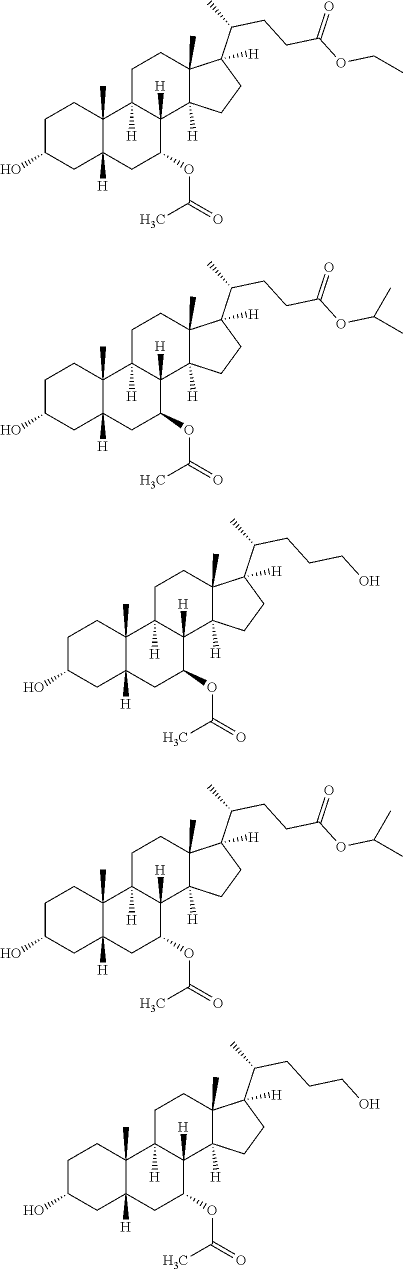 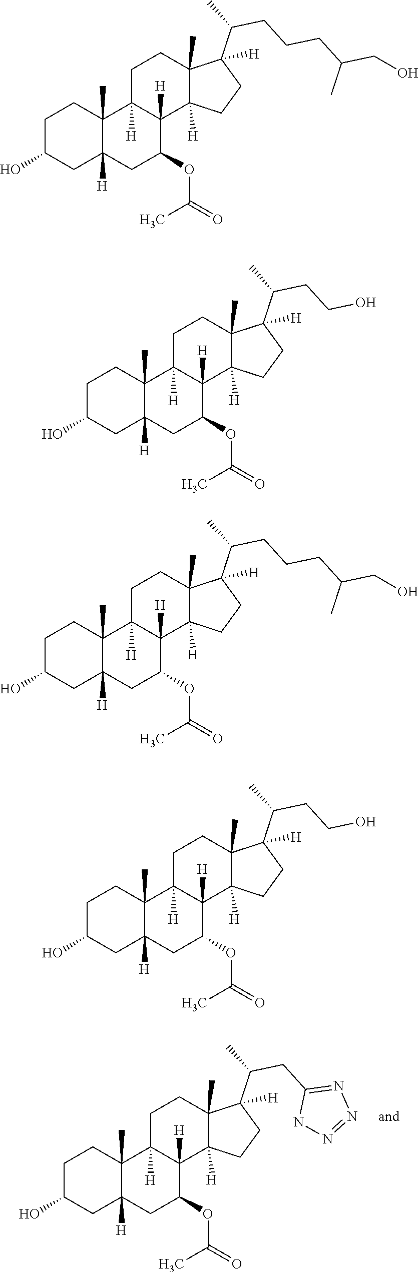 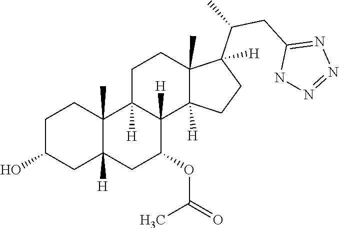
and salts thereof.

16. A compound of formula (I) as described in claim 15 or a salt thereof, wherein:
R¹ is (C₁-C₆)alkyl that is substituted with one or more R⁵, wherein R⁵ is independently selected from the group consisting of halo, cyano, heterocycle, heteroaryl, —OH, —C(═O)NH(C₁-C₆ alkyl)(S(O)₂R^{c}), —C(═O)NH(C₁-C₆ alkyl)(C(═O)OR^{c}), —C(═O)NR^{a}R^{b}, —OS(O)₃R^{c}, —C(═O)NH(S(O)₂R^{c}), —C(═O)OR^{c} and —C(═O)(C₁-C₆ alkyl), wherein any heterocycle and heteroaryl is optionally substituted with one or more groups independently selected from the group consisting of halo, carboxy, —OH, cyano, (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy, wherein any (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy is optionally substituted with one or more groups independently selected from the group consisting of —OH and (C₁-C₆)alkoxy;
R² is selected from the group consisting of —OH, —OC(═O)R^{c}, —OS(O)₃H and (C₁-C₆)alkoxy;
R³ is (C₁-C₃)alkoxy;
R⁴ is selected from the group consisting of H and —OH, where;
R^{a} and R^{b} are independently selected from H and (C₁-C₆)alkyl that is optionally substituted with one or more groups independently selected from the group consisting of hydroxy and (C₁-C₆)alkoxy that is optionally substituted with one or more groups independently selected from the group consisting of hydroxy and (C₁-C₆)alkoxy; or R^{a} and R^{b} together with the nitrogen to which they are attached form a 4-6 membered ring heterocycle;
each R^{c} is independently selected from H and (C₁-C₆)alkyl; and
R⁶ is selected from the group consisting of H and (C₁-C₆)alkyl, or R⁶ and R³ together with the atom to which they are attached form a carbocycle or heterocycle, wherein the carbocycle or heterocycle is optionally substituted with halo, cyano, heteroaryl, —OH and (C₁-C₆)alkyl;
provided that the compound or salt is not selected from the group consisting of: 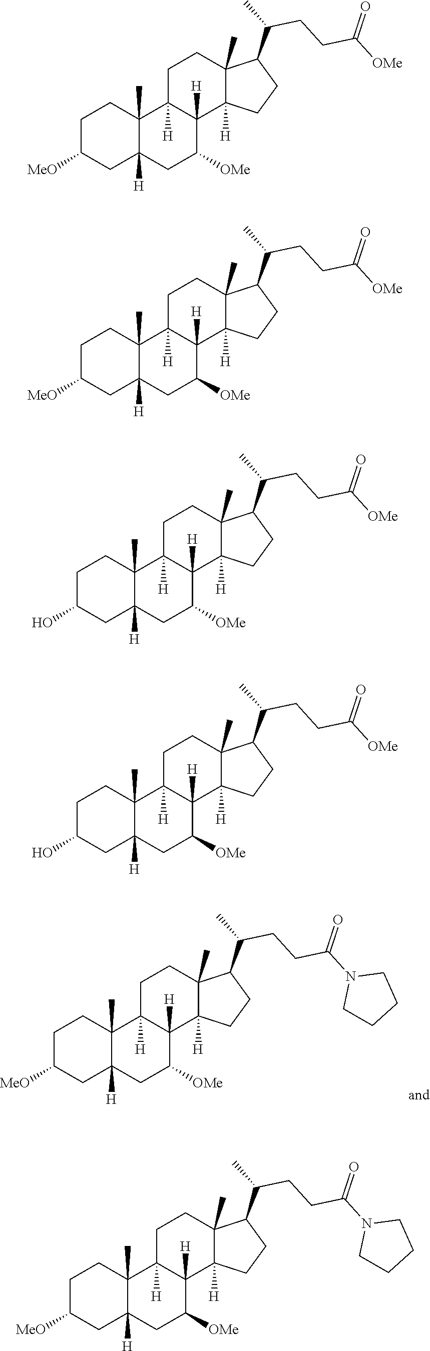
and salts thereof.

17. A compound of formula (I) as described in claim 15 or a salt thereof, wherein:
R¹ is (C₁-C₆)alkyl that is substituted with one or more R⁵, wherein R⁵ is independently selected from the group consisting of halo, cyano, heterocycle, heteroaryl, —OH, —C(═O)NH(C₁-C₆ alkyl)(S(O)₂R^{c}), —C(═O)NH(C₁-C₆ alkyl)(C(═O)OR^{c}), —C(═O)NR^{a}R^{b}, —OS(O)₃R^{c}, —C(═O)NH(S(O)₂R^{c}), —C(═O)OR^{c}, and —C(═O)(C₁-C₆ alkyl), wherein any heterocycle and heteroaryl is optionally substituted with one or more groups independently selected from the group consisting of halo, carboxy, —OH, cyano, (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy, wherein any (C₁-C₆)alkyl, (C₃-C₆)cycloalkyl, (C₁-C₆)alkoxy, (C₂-C₆)alkenyl, (C₂-C₆)alkynyl, (C₁-C₆)alkanoyl, (C₁-C₆)alkoxycarbonyl, (C₁-C₆)alkylthio and (C₂-C₆)alkanoyloxy is optionally substituted with one or more groups independently selected from the group consisting of —OH and (C₁-C₆)alkoxy;
R² is —OH;
R³ is —OC(═O)H;
R⁴ is selected from the group consisting of H and —OH, where
R^{a} and R^{b} are independently selected from H and (C₁-C₆)alkyl that is optionally substituted with one or more groups independently selected from the group consisting of hydroxy and (C₁-C₆)alkoxy that is optionally substituted with one or more groups independently selected from the group consisting of hydroxy and (C₁-C₆)alkoxy; or R⁶ and R³ together with the nitrogen to which they are attached form a 4-6 membered ring heterocycle;
each R^{c} is independently selected from H and (C₁-C₆)alkyl; and
R⁶ is selected from the group consisting of H and (C₁-C₆)alkyl, or R⁶ and R³ together with the atom to which they are attached form a carbocycle or heterocycle, wherein the carbocycle or heterocycle is optionally substituted with halo, cyano, heteroaryl, —OH and (C₁-C₆)alkyl;
provided that the compound or salt is not selected from the group consisting of: 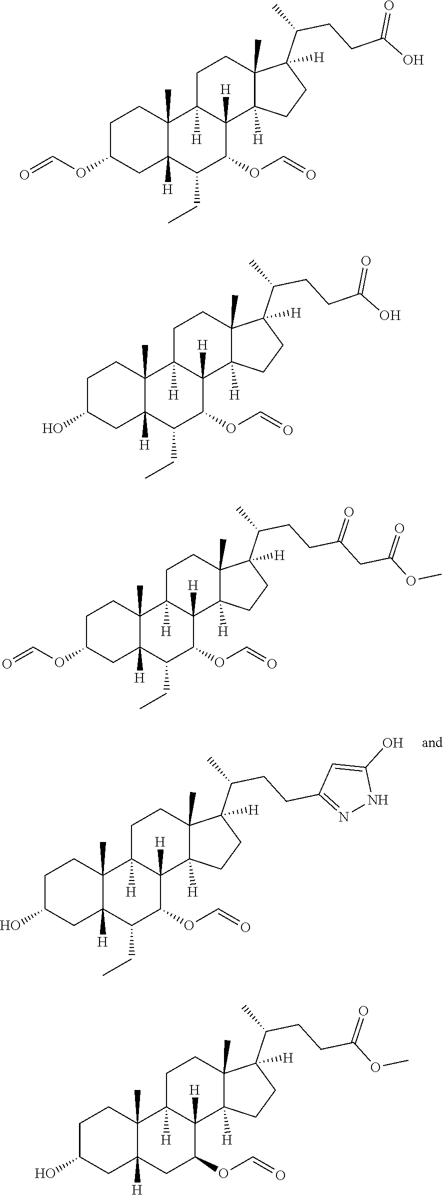
and salts thereof.

18. The compound of claim 14 wherein R^{d} is H or (C₂-C₆)alkyl.

19. The compound of claim 14 wherein R² is —OH, and R^{d} is H or (C₁-C₆)alkyl.

20. A pharmaceutical composition comprising a compound as described in claim 14 or a pharmaceutically acceptable salt thereof, and a pharmaceutically acceptable excipient.
